(19) United States
(12) Patent Application Publication
(10) Pub. No.: US 2023/0372539 A1
(43) Pub. Date: Nov. 23, 2023

(54) VIRAL VECTORS ENCODING GLP-1 RECEPTOR AGONIST FUSIONS AND USES THEREOF IN TREATING METABOLIC DISEASES

(71) Applicant: The Trustees of the University of Pennsylvania, Philadelphia, PA (US)

(72) Inventors: James M. Wilson, Philadelphia, PA (US); Christian Hinderer, Baltimore, MD (US); Makoto Horiuchi, Wallingford, PA (US)

(73) Assignee: The Trustees of the University of Pennsylvania, Philadelphia, PA (US)

(21) Appl. No.: 18/042,726

(22) PCT Filed: Aug. 24, 2021
(86) PCT No.: PCT/US2021/047411

§ 371 Date: Feb. 23, 2023

Related U.S. Application Data
(60) Provisional application No. 63/069,500, filed on Aug. 24, 2020.

(51) Int. Cl. A61K 48/00; C12N 15/86; C07K 14/605

(52) U.S. Cl. CPC A61K 48/005; C12N 15/86; C07K 14/605; C12N 2750/14143

(57) ABSTRACT

Compositions and methods for treating metabolic diseases in a subject are provided. A viral vector is provided which includes a nucleic acid molecule comprising a sequence encoding a GLP-1 receptor agonist fusion protein and regulatory sequences which direct expression thereof.

### BACKGROUND OF THE INVENTION

Glucagon-like peptide 1 (GLP-1) is an endogenous peptide hormone that plays a central role in glucose homeostasis. GLP-1 is a peptide hormone that is produced in the gastrointestinal (GI) tract, from proteolytic cleavage of glucagon pre-protein. GLP-1 and other GLP-1 receptor agonists have the ability to control hyperglycemia by potentiating insulin release, increasing insulin sensitivity, preventing beta cell loss, and delaying gastric emptying. However, GLP-1 has a short half-life, which has prevented its use as a drug. Other GLP-1 receptor agonists are currently used in humans for the treatment of diabetes. GLP-1 receptor agonists engineered to overcome the short half-life of the native hormone by fusing the agonist to a protein with a longer half-life have emerged as important therapeutics for the treatment of type 2 diabetes mellitus (T2DM).

### SUMMARY OF THE INVENTION

Viral vectors encoding glucagon-like peptide 1 (GLP-1) receptor agonist fusion protein constructs are provided herein. These viral vectors may achieve, in some embodiments, sustained expression of the GLP-1 receptor agonist in subjects and/or increased circulating half-life, as compared to vector-mediated delivery of a GLP-1 receptor agonist without a fusion partner. Further provided are methods of making and using such viral vectors.

In one aspect, a viral vector is provided which includes a nucleic acid comprising a polynucleotide sequence encoding a fusion protein. The fusion protein includes (a) a leader sequence comprising a secretion signal peptide, (b) a glucagon-like peptide-1 (GLP-1) receptor agonist, and (c) a fusion domain comprising either (i) an IgG Fc or a functional variant thereof or (ii) an albumin or a functional variant thereof. In one embodiment, the vector is an adeno-associated viral vector.

In on embodiment, the (i) the secretion signal peptide of the leader sequence comprises a thrombin signal peptide; (ii) the leader sequence comprises a thrombin propeptide; and/or (iii) the leader sequence comprises a thrombin leader sequence. In another embodiment, the leader sequence comprises an IL-2 leader sequence. In one embodiment, the GLP-1 receptor agonist is selected from SEQ ID NO: 1, SEQ ID NO: 2, SEQ ID NO: 3, SEQ ID NO: 4, SEQ ID NO: 5, SEQ ID NO: 6, and functional variants thereof.

In one embodiment, the fusion domain is a human IgG4 Fc having the sequence of SEQ ID NO: 11, or a sequence sharing at least 90% identity therewith, or a functional variant thereof. In another embodiment, the fusion domain is a human albumin having the sequence of SEQ ID NO: 12, or a sequence sharing at least 90% identity therewith, or a functional variant thereof. In one embodiment, the fusion domain is a rhesus IgG4 Fc having the sequence of SEQ ID NO: 17, or a sequence sharing at least 90% identity therewith, or a functional variant thereof.

In another aspect, the viral vector includes an AAV capsid, and a vector genome packaged in the AAV capsid, said vector genome comprising AAV inverted terminal repeats (ITRs), the polynucleotide sequence encoding the fusion protein, and regulatory sequences which direct expression of the fusion protein.

In another aspect, a pharmaceutical composition suitable for use in treating a metabolic disease in a subject is provided. The composition includes an aqueous liquid and the viral vector as described herein. In one embodiment, the subject is a human.

In yet another aspect, use of a viral vector as described herein is provided for the manufacture of a medicament for treating a subject having a metabolic disease, optionally diabetes.

In another aspect, a method of treating a subject having a metabolic disease is provided. The method includes administering to the subject an effective amount of a viral vector or composition as described herein,

Other aspects and advantages of the invention will be readily apparent from the following detailed description of the invention.

### BRIEF DESCRIPTION OF THE DRAWINGS

FIG. 1A is a schematic drawing of Dulaglutide.

FIG. 1B is a schematic drawing of Albiglutide.

FIG. 2 shows inducible hDulaglutide(Trb) vs CB7.feDulaglutide(feTrb) in vitro. GLP1-Fc fusions were measured in culture supernatants of HEK293 cells transfected with plasmids for inducible human Dulaglutide with human Thrombin signal sequence (TF.GT2A.Dulaglutide(Trb)) and CB7.feline Dulaglutide (feTrb). Supernatants were collected at 48 hr after treatment with Rapamycin (Rapa) at 0, 4, and 40 nM or at 48 hr after transfection for CB7.feDulaglutide(feTrb). GLP1-Fc was quantified by active form GLP1 ELISA along with kit's STD.

FIG. 3 shows inducible expression of GLP-1 in Rag1KO (RAG1^{-/-}) mice (n=5/vector). Rag1KO female mice were dosed with 1×10¹¹ GC/mouse via intramuscular (I.M. or IM) delivery of the shown vectors (i.e., AAVrh91.TF.hDulaglutide(Trb)3w.rBG and AAVrh91.TF.rhDulaglutide(rhTrb).3w.rBG). Weekly bleeds were performed. GLP1 ELISA specific for active form of GLP-1 was performed. AAV Vectors were injected at day 0 and rapamycin administered by oral gavage around days 14 and 15 post AAV injection.

FIG. 4 is a schematic of a plasmid map of pAAV.CMV.TF.GT2A. Dulaglutide(Trb).3w.rBG.

FIG. 5 shows AAV-mediated expression of engineered GLP-1 construct in mice.

FIG. 6A shows a schematic of an example expression cassette comprising inducible construct for use in a two-vector system.

FIG. 6B shows a schematic of an expression cassette comprising an inducible construct for use in a 1-vector system, comprising an IRES linker.

FIG. 7A shows a schematic of an expression cassette comprising an inducible construct for use in a 1-vector system, comprising an F2A cleavage sequence linker and human GLP1-Fc (hDulaglutide) with secretory signal.

FIG. 7B shows a further detailed view of a GT2A cleavage sequences, wherein GT2A_V1 comprises an amino acid sequence of SEQ ID NO: 21, and GT2A_V2 comprises an amino acid sequence of SEQ ID NO: 22.

FIG. 8 shows expression of rhesus monkey exemplary therapeutic transgene (rhTT) in HEK293 cell supernatant as measured following transfection with various constructs and treatment with Rapamycin at 0 nM, 4 nM, and 40 nM, and plotted as IU/mL of rhTT.

FIG. 9 shows inducible human (h) and rhesus macaque (rh) GLP-1 expression in vitro. GLP1-Fc fusions were measured in culture supernatants of HEK293 cells transfected with plasmids for inducible hDulaglutide comprising Thrombin signal sequence, rhDulaglutide comprising 2-vector system, and CB7.rhDulaglutide. Cell were plated on Day 0, transfected in Day 1, treated with Rapamycin at 0 nM, 4 nM, and 40 nM on Day 2, and supernatants from cells were collected on Day 4 or at 48 hr after transfection for CB7.rhDulaglutide(rhTrb). GLP1-Fc was quantified by active form GLP1 ELISA along with kit's STD.

FIGS. 10A to 10C show rhGLP1-Fc expression and analysis of an anti-rhGLP1-Fc ADA (anti-drug antibody) detection assay for NHP1 (18-128). FIG. 10A shows rhGLP1-Fc expression levels in serum plotted as nM, as measured on days 0 to 200. FIG. 10B shows rapamycin levels in serum plotted as μg/L, as measured on days 0 to 200. FIG. 10C shows results of an ADA detection assay plotted as O.D. 450 nm, as measured on days 0 to 200.

FIGS. 11A to 11C show rhGLP1-Fc expression and analysis of an anti-rhGLP1-Fc ADA assay for NHP1 (18-072). FIG. 11A shows rhGLP1-Fc expression levels in serum plotted as nM, as measured on days 0 to 200. FIG. 11B shows rapamycin levels in serum plotted as μg/L, as measured on days 0 to 200. FIG. 11C shows results of an ADA detection assay plotted as O.D. 450 nm, as measured on days 0 to 200.

FIGS. 12A to 12C show rhGLP1-Fc expression and analysis of an anti-rhGLP1-Fc ADA assay for NHP1 (18-013). FIG. 12A shows rhGLP1-Fc expression levels in serum plotted as nM, as measured on days 0 to 200. FIG. 12B shows rapamycin levels in serum plotted as μg/L, as measured on days 0 to 200. FIG. 12C shows results of an ADA detection assay plotted as O.D. 450 nm, as measured on days 0 to 200.

### DETAILED DESCRIPTION OF THE INVENTION

Long-acting GLP-1 receptor agonist fusion protein expression constructs have been developed for use in subjects in need thereof, including humans. A leader sequence is provided which includes a secretion signal peptide, as well as a fusion domain which is intended to prolong the time in circulation of the resulting fusion protein.

Delivery of these constructs to subjects in need thereof via a number of routes, and particularly by expression in vivo mediated by a recombinant vector such as a rAAV vector, is described. Also provided are methods of using these constructs in regimens for treating diabetes or metabolic syndrome in a subject in need thereof and increasing the half-life of GLP-1 in a subject. In addition, methods are provided for enhancing the activity of GLP-1 in a subject. Also provided are methods for inducing weight loss in a subject in need thereof.

### GLP-1 Fusion Proteins

Glucagon-like peptide 1, or GLP-1, is an incretin derived from the transcription product of the proglucagon gene. In vivo, the glucagon gene expresses a 180 amino acid prepropolypeptide that is proteolytically processed to form glucagon, two forms of GLP-1 and GLP-2. The original sequencing studies indicated that GLP-1 possessed 37 amino acid residues. However, subsequent information showed that this peptide was a propeptide and was additionally processed to remove 6 amino acids from the amino-terminus to a form GLP-1 (7-37), an active form of GLP-1. The glycine at position 37 is also transformed to an amide in vivo to form GLP-1 (7-36) amide. GLP-1 (7-37) and GLP-1 (7-36) amide are insulinotropic hormones of equal potency. Thus, as used herein, the biologically “active” forms of GLP-1 which are useful herein are: GLP-1-(7-37) and GLP-1-(7-36)NH₂.

GLP-1 receptor agonists are a class of antidiabetic agents that mimic the action of the glucagon-like peptide. GLP-1 is one of several naturally occurring incretin compounds that affect the body after they are released from the gut during digestion. By binding and activating GLP-1 receptors, GLP-1 receptor agonists are able to reduce blood glucose levels helping T2DM patients to reach a glycemic control. As used herein the term “GLP-1 receptor agonist” refers to at least a GLP-1 or a functional fragment thereof, amino-acid sequence variants of GLP-1 or functional fragments thereof, and other polypeptide agonists for the GLP-1 receptor (e.g., exedin-4 and variants thereof). The disclosure provides fusion proteins comprising one or more copies of a GLP-1 receptor agonist, as well as polynucleotides and vectors encoding such fusion proteins. In some embodiments, the fusion protein comprises a polynucleotide sequence encoding a fusion protein comprising (a) a leader sequence comprising a secretion signal peptide, (b) a glucagon-like peptide-1 (GLP-1) receptor agonist, and (c) a fusion domain. In one embodiment, the GLP-1 receptor agonist comprises a thrombin leader sequence, a GLP-1 receptor agonist, and an IgG Fc or functional variant thereof. In another embodiment, the fusion protein comprises a thrombin leader, a GLP-1 receptor agonist, and an albumin or functional variant thereof. In another embodiment, the fusion protein comprises a thrombin leader, two copies of a GLP-1 receptor agonist, and an albumin or functional variant thereof.

In some embodiments, GLP-1 receptor agonists include variants which may include up to about 10% variation from a GLP-1 nucleic acid or amino acid sequence described herein or known in the art, which retain the function of the wild type sequence. As used herein, by “retain function” it is meant that the nucleic acid or amino acid functions in the same way as the wild type sequence, although not necessarily at the same level of expression or activity. For example, in one embodiment, a functional variant has increased expression or activity as compared to the wild type sequence. In another embodiment, the functional variant has decreased expression or activity as compared to the wild type sequence. In one embodiment, the functional variant has 10%, 20%, 30%, 40%, 50%, 60%, 70%, 80%, 90% or greater increase or decrease in expression or activity as compared to the wild type sequence.

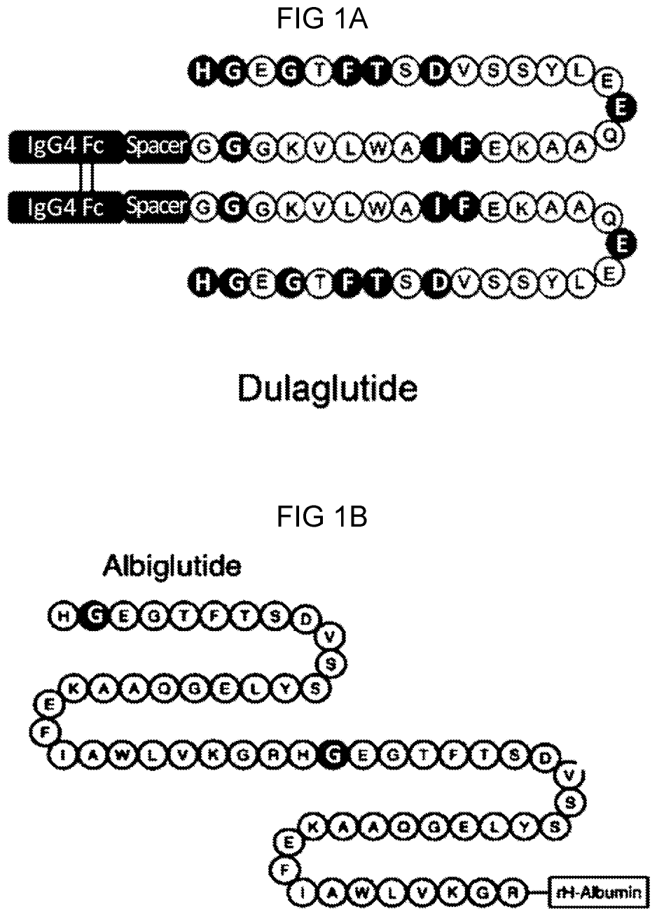

Several human drugs that fuse a GLP-1 receptor agonist to a stabilizing fusion domain are known in the art. These include, albiglutide, liraglutide, dulaglutide and lixisenatide (also known by its chemical name des-38-proline-exendin-4 (Heloderma suspectum)-(1-39)-peptidylpenta-L-lysyl-L-lysinamide). Dulaglutide is a disulfide-bonded homodimer fusion peptide with each monomer consisting of one GLP-1 analog moiety and one IgG4 Fc region. Yu M, et al. (2018) Battle of GLP-1 delivery technologies, Adv. Drug Deliv. Rev. A schematic of dulaglutide is shown in FIG. 1A. See, WO 2005/000892A2, which is incorporated herein by reference.

Albiglutide is a recombinant protein composed of two copies of GLP-1 analogs fused to human albumin. The molecule has a Gly8 to Ala substitution in both copies of the GLP-1 analogs to improve resistance to DPP-4 degradation. A schematic of albiglutide is shown in FIG. 1B.

The fusion comprises, in one embodiment, a GLP-1 analog in combination with heterologous sequences. By GLP-1 analog is meant a polypeptide sharing at least 90%, 95%, 97%, 98%, 99% or 100% identity with native human GLP-1(7-37). In one embodiment, the GLP-1 analog has at most 1, 2, or 3 amino acid substitutions as compared to the native sequence. Native human GLP-1(1-37) has the sequence of HDEFERHAEGTFTSDVSSYLEGQAAKEFIAWLVKGRG (SEQ ID NO: 1), with GLP-1(7-37) having the sequence of HAEGTFTSDVSSYLEGQAAKEFIAWLVKGRG (SEQ ID NO: 2). In some embodiments, it is desirable to alter the native GLP-1 sequence to optimize one or more features thereof. For example, in one embodiment, the GLP-1 analog contains one, two, or three amino acid substitutions selected from A8G, G22E, and R36G as compared to the native sequence. These substitutions have been shown to improve efficacy of the clinical profile of GLP-1, including protection from DPP-4 inactivation (A8G), increased solubility (G22E), and reduction of immunogenicity via substituting a glycine residue for arginine at position 36 (R36G) to remove a potential T-cell epitope. In one embodiment, the GLP-1 analog is a DPP-IV resistant variant of GLP-1. In one embodiment, the GLP-1 analog has a sequence comprising, or consisting of, SEQ ID NO: 3: HGEGTFTSDVSSYLEEQAAKEFIAWLVKGGG. In another embodiment, the GLP-1 analog has a sequence comprising, or consisting of, SEQ ID NO: 4: HGEGTFTSDVSSYLEGQAAKEFIAWLVKGRG. In another embodiment, the GLP-1 receptor agonist has a sequence comprising, or consisting, of SEQ ID NO: 5: HGEGTFTSDLSKQMEEEAVRLFIEWLKNGGPSSGAPPPS or a functional variant thereof. In one embodiment, the variant shares at least 90% identity, 95% identity, 97% identity, 98% identity, 99% identity or 100% identity with SEQ ID NO: 5. In another embodiment, the GLP-1 receptor agonist has a sequence comprising, or consisting, of SEQ ID NO: 6: HGEGTFTSDLSKQMEEEAVRLFIEWLKNGGPSSGAPPSKKKKKK or a functional variant thereof. In one embodiment, the variant shares at least 90% identity, 95% identity, 97% identity, 98% identity, 99% identity or 100% identity with SEQ ID NO: 6. In one embodiment, more than one copy of the GLP-1 analog is present in the fusion protein. In another embodiment, the GLP-1 receptor agonist is two tandem copies of GLP-1(7-37) or a DPP-IV resistant variant thereof.

The fusion protein may comprise a leader sequence, which may comprise a secretion signal peptide. As used herein, the term “leader sequence” refers to any N-terminal sequence of a polypeptide.

The leader sequence may be derived from the same species for which administration is ultimately intended, e.g., a human. As used herein, the terms “derived” or “derived from” mean the sequence or protein is sourced from a specific subject species or shares the same sequence as a protein or sequence sourced from a specific subject species. For example, a leader sequence which is “derived from” a human, shares the same sequence (or a variant thereof, as defined herein) as the same leader sequence as expressed in a human. However, the specified nucleic acid or amino acid need not actually be sourced from a human. Various techniques are known in the art which are able to produce a desired sequence, including mutagenesis of a similar protein (e.g., a homolog) or artificial production of a nucleic acid or amino acid sequence. The “derived” nucleic acid or amino acid retains the function of the same nucleic acid or amino acid in the species from which it is “derived”, regardless of actual source of the derived sequence.

The term “amino acid substitution” and its synonyms are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

In one embodiment, the leader is a human thrombin (Factor II) sequence. In one embodiment, the thrombin leader has the sequence shown in SEQ ID NO: 7: MAHVRGLQLPGCLALAALCSLVHSQHVFLAPQQARSLLQRVRR, or a functional variant thereof having at most 1, 2, or 3 amino acid substitutions. In some embodiments, the leader comprises a signal peptide and a propeptide. In one embodiment, the secretion signal peptide of the leader sequence comprises a human thrombin signal peptide. In one embodiment, the signal peptide is MAHVRGLQLPGCLALAALCSLVHS (SEQ ID NO: 8) or a functional variant thereof having at most 1, 2, or 3 amino acid substitutions. In another embodiment, the leader sequence comprises a human thrombin propeptide. In one embodiment, the propeptide has the sequence of QHVFLAPQQARSLLQRVRR (SEQ ID NO: 9) or a functional variant thereof having at most 1, 2, or 3 amino acid substitutions.

In one embodiment, the leader is a human IL-2 sequence. In one embodiment, the IL-2 leader has the sequence shown in SEQ ID NO: 10: MYRMQLLSCIALSLALVTNS, or a functional variant thereof having at most 1, 2, or 3 amino acid substitutions.

In one embodiment, functional variants of the desired leader include variants which may include up to about 10% variation from a leader nucleic acid or amino acid sequence described herein or known in the art, which retain the function of the wild type sequence.

In some embodiments, the coding regions for both the propeptide and GLP-1 peptide are incorporated into a single nucleic acid sequence without a linker between the coding sequences of the propeptide and GLP-1.

The fusion protein further includes a fusion domain. The fusion domain, in one embodiment, is a human IgG Fc fragment or a functional variant thereof. Immunoglobulins typically have long circulating half-lives in vivo. By fusing the GLP-1 receptor agonist (and leader) to an IgG Fc, the circulation time of the fusion protein is prolonged, while the function of the GLP-1 is preserved. In another embodiment, the fusion domain is a rhesus IgG Fc fragment or functional variant thereof.

As used herein, the Fc portion of an immunoglobulin has the meaning commonly given to the term in the field of immunology. Specifically, this term refers to an antibody fragment which does not contain the two antigen binding regions (the Fab fragments) from the antibody. The Fc portion consists of the constant region of an antibody from both heavy chains, which associate through non-covalent interactions and disulfide bonds. The Fc portion can include the hinge regions and extend through the CH2 and CH3 domains to the c-terminus of the antibody. The Fc portion can further include one or more glycosylation sites. In one embodiment, the fusion domain is a human IgG Fc. The four subclasses, IgG1, IgG2, IgG3, and IgG4, which are highly conserved, differ in their constant region, particularly in their hinges and upper CH2 domains. See, Vidarsson et al, IgG Subclasses and Allotypes: From Structure to Effector Functions, Front Immunol. October 2014; 5: 520, which is incorporated herein by reference. The Fc domain can be derived from any human IgG, including human IgG1, human IgG2, human IgG3, or human IgG4. In one embodiment, the human IgG Fc is an IgG4 Fc. In one embodiment, the human IgG Fc is SEQ ID NO: 11: AESKYGPPCPPCPAPEAAGGPSVFLFPPKPKDTLMISRTPEVTCVVVDVSQEDP EVQFNWYVDGVEVHNAKTKPREEQFNSTYRVVSVLTVLHQDWLNGKEYKCKVSN KGLPSSIEKTISKAKGQPREPQVYTLPPSQEEMTKNQVSLTCLVKGFYPSDIAVEWES NGQPENNYKTTPPVLDSDGSFFLYSRLTVDKSRWQEGNVFSCSVMHEALHNHYTQK SLSLSLG. In another embodiment, the human IgG Fc shares at least 90% identity, at least 95% identity, at least 99% identity, or at least 100% identity to SEQ ID NO: 11.

In another embodiment, the fusion domain is a rhesus IgG Fc. The Fc domain can be derived from any rhesus IgG, including rhesus IgG1, rhesus IgG2, rhesus IgG3, or rhesus IgG4. In one embodiment, the rhesus IgG Fc is an IgG4 Fc. In one embodiment, the rhesus IgG Fc is SEQ ID NO: 17:

PPCPPCPAPE LLGGPSVFLF PPKPKDTLMI SRTPEVTCVV VDVSQEDPEV QFNWYVDGVE VHNAQTKPRE RQFNSTYRVV SVLTVTHQDW LNGKEYTCKV SNKGLPAPIE KTISKAKGQP REPQVYILPP PQEELTKNQV SLTCLVTGFY PSDIAVEWES NGQPENTYKT TPPVLDSDGS YLLYSKLTVN KSRWQPGNIF TCSVMHEALH NHYTQKSLSV SPGK. In another embodiment, the rhesus IgG Fc shares at least 90% identity, at least 95% identity, at least 99% identity, or at least 100% identity to SEQ ID NO: 17. In one embodiment, the rhesus IgG further comprises a hinge sequence.

In another embodiment, the fusion domain is a human albumin or a functional variant thereof. In one embodiment, the human albumin is SEQ ID NO: 12: DAHKSEVAHRFKDLGEENFKALVLIAFAQYLQQCPFEDHVKLVNEVTEFAKTCVAD ESAENCDKSLHTLFGDKLCTVATLRETYGEMADCCAKQEPERNECFLQHKDDNPNL PRLVRPEVDVMCTAFHDNEETFLKKYLYEIARRHPYFYAPELLFFAKRYKAAFTECC QAADKAACLLPKLDELRDEGKASSAKQRLKCASLQKFGERAFKAWAVARLSQRFP KAEFAEVSKLVTDLTKVHTECCHGDLLECADDRADLAKYICENQDSISSKLKECCEK PLLEKSHCIAEVENDEMPADLPSLAADFVESKDVCKNYAEAKDVFLGMFLYEYARR HPDYSVVLLLRLAKTYETTLEKCCAAADPHECYAKVFDEFKPLVEEPQNLIKQNCEL FEQLGEYKFQNALLVRYTKKVPQVSTPTLVEVSRNLGKVGSKCCKHPEAKRMPCAE DYLSVVLNQLCVLHEKTPVSDRVTKCCTESLVNRRPCFSALEVDETYVPKEFNAETF TFHADICTLSEKERQIKKQTALVELVKHKPKATKEQLKAVMDDFAAFVEKCCKADD KETCFAEEGKKLVAASQAALGL. In another embodiment, the human albumin shares at least 90% identity, at least 95% identity, at least 99% identity, or at least 100% identity to SEQ ID NO: 12.

The in vivo function and stability of the fusion proteins of the present disclosure may be optimized by adding small peptide linkers, e.g., to prevent potentially unwanted domain interactions or for other reasons. Further, a glycine-rich linker may provide some structural flexibility such that the GLP-1 analog portion can interact productively with the GLP-1 receptor on target cells such as the beta cells of the pancreas. Thus, the C-terminus of the GLP-1 analog and the N-terminus of the fusion domain of the fusion protein are, in one embodiment, fused via a linker. In one embodiment, the linker includes 1, 1.5 or 2 repeats of a G-rich peptide linker having the sequence GGGGSGGGGSGGGGS (SEQ ID NO: 13).

In one embodiment, the fusion protein comprises (a) human thrombin leader, (b) a DPP-IV resistant variant of GLP-1(7-37), a linker, and (c) a human IgG Fc. In one embodiment, the fusion protein has the sequence of SEQ ID NO: 14, or a sequence at least 90%, at least 95%, at least 98%, or at least 99% identical thereto.

SEQ ID NO: 14
MAHVRGLQLPGCLALAALCSLVHSQHVFLAPQQARSLLQRVRRHGEGTF
TSDVSSYLEEQAAKEFIAWLVKGGGGGGGSGGGGSGGGGSAESKYGPPC
PPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNV
FSCSVMHEALHNHYTQKSLSLSLG

In one embodiment, the sequence encoding the fusion protein is SEQ ID NO: 15 or a sequence at least 75%, at least 85%, at least 90%, at least 95%, at least 98%, or at least 99% identical thereto.

SEQ ID NO: 15:
atggctcacgttcgaggactgcagctgcctggatgtctggctcttgccg
ctctgtgtagcctggtgcacagccagcacgtgtttctggctcctcagca
agccagatcactgctgcagagagttagaaggcacggcgagggcaccttt
acctccgacgtgtctagctacctggaagaacaggccgccaaagagttta
tcgcctggctggtcaaaggtggcggcggaggcggaggaagcggtggcgg
aggttcaggtggtggtggatctgccgagtctaagtacggccctccttgt
cctccctgtcctgctcccgaagctgctggcggcccatccgtgtttctgt
tccctccaaagcctaaggacaccctgatgatcagcagaacccctgaagt
gacctgcgtggtggtcgacgtgtcccaagaggatcctgaggtgcagttc
aattggtacgtggacggcgtggaagtgcacaacgccaagaccaagccta
gagaggaacagttcaacagcacctacagagtggtgtccgtgctgaccgt
gctgcaccaggattggctgaacggcaaagagtacaagtgcaaggtgtcc
aacaagggcctgcctagctccatcgagaaaaccatcagcaaggccaagg
gccagccaagagaaccccaggtgtacacactgcctccaagccaagagga
aatgaccaagaaccaggtgtccctgacctgcctcgtgaagggcttctac
ccttccgatatcgccgtggaatgggagagcaatggccagcctgagaaca
actacaagaccacacctcctgtgctggacagcgacggctcattcttcct
gtacagcagactgaccgtggacaagagcagatggcaagagggcaacgtg
ttcagctgcagcgtgatgcacgaggccctgcacaaccactacacccaga
agtctctgagcctgagcctgggc

In one embodiment, the fusion protein comprises (a) human thrombin leader, (b) a DPP-IV resistant variant of GLP-1(7-37), a linker, and (c) a rhesus IgG Fc. In one embodiment, the fusion protein comprises (a) rhesus thrombin leader, (b) a DPP-IV resistant variant of GLP-1(7-37), a linker, and (c) a rhesus IgG Fc.

In one embodiment, the fusion protein has the sequence of SEQ ID NO: 37, or a sequence at least 90%, at least 95%, at least 98%, or at least 99% identical thereto.

SEQ ID NO: 37
MAHVRGLQLPGCLALAALCSLVHSQHVFLAPQQALSLLQRVRRHGEGTF
TSDVSSYLEEQAAKEFIAWLVKGGGGGGGSGGGGSGGGGSAEFTPPCPP
CPAPELLGGPSVFLFPPKPKDTLMISRTPEVTCVVVDVSQEDPEVQFNW
YVDGVEVHNAQTKPRERQFNSTYRVVSVLTVTHQDWLNGKEYTCKVSNK
GLPAPIEKTISKAKGQPREPQVYILPPPQEELTKNQVSLTCLVTGFYPS
DIAVEWESNGQPENTYKTTPPVLDSDGSYLLYSKLTVNKSRWQPGNIFT
CSVMHEALHNHYTQKSLSVSPG

In one embodiment, the sequence encoding the fusion protein is SEQ ID NO: 36 or a sequence at least 75%, at least 85%, at least 90%, at least 95%, at least 98%, or at least 99% identical thereto.

SEQ ID NO: 36
atggctcacgttcgaggactgcagctgcctggatgtctggctcttgccg
ctctgtgtagcctggtgcacagccagcatgtgtttctggctcctcaaca
agccctgagcctgctgcaaagagttagaaggcacggcgagggcaccttc
acctccgacgtgtccagctacctggaagaacaggccgccaaagagttta
tcgcctggctggtcaaaggcggtggtggtggcggaggatctggcggagg
tggaagcggcggaggcggatctgctgagtttacacctccttgtcctccc
tgtcctgctcccgagctgctcggaggcccttccgtgtttctgttccctc
caaagcctaaggacaccctgatgatcagcagaacccctgaagtgacctg
cgtggtcgtggacgtgtcccaagaggatcctgaggtgcagttcaattgg
tacgtggacggcgtggaagtgcacaacgcccagacaaagcccagagagc
ggcagttcaacagcacctacagagtggtgtccgtgctgaccgtgacaca
ccaggattggctgaacggcaaagagtacacctgtaaagtctccaacaag
ggcctgcctgctcctatcgagaaaaccatcagcaaggccaagggccagc
ctagagaaccccaggtgtacatcctgcctccacctcaagaggaactgac
caagaaccaggtgtccctgacctgtctggtcaccggcttctacccttcc
gatatcgccgtggaatgggagagcaacggacagcccgagaacacctaca
agaccacacctccagtgctggacagcgacggcagctatctgctgtactc
caagctgacagtgaacaagagccggtggcagcccggcaacatcttcacc
tgttctgtgatgcacgaggccctgcacaaccactacacccagaagtctc
tgagcgtcagccctggc

In one embodiment, the fusion protein comprises (a) human thrombin leader, (b) a DPP-IV resistant variant of GLP-1(7-37), a linker, and (c) a human albumin. In another embodiment, the fusion protein comprises fusion protein comprises (a) human thrombin leader, (b) two tandem copies of human GLP-1(7-37) or a DPP-IV resistant variant thereof, a linker, and (c) a human albumin.

When a variant or fragment of the leader sequence, GLP-1 receptor agonist, or fusion domain is desired, the coding sequences for these peptides may be generated using site-directed mutagenesis of the wild-type nucleic acid sequence. Alternatively or additionally, web-based or commercially available computer programs, as well as service based companies may be used to back translate the amino acids sequences to nucleic acid coding sequences, including both RNA and/or cDNA. See, e.g., backtranseq by EMBOSS, ebi.ac.uk/Tools/st/; Gene Infinity (geneinfinity.org/sms-/sms_backtranslation.html); ExPasy (expasy.org/tools/). In one embodiment, the RNA and/or cDNA coding sequences are designed for optimal expression in the subject species for which administration is ultimately intended, e.g., a human.

The coding sequences may be designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In addition to the leader sequences, GLP-1 receptor agonists, fusion domains, and fusion proteins provided herein, nucleic acid sequences encoding these polypeptides are provided. In one embodiment, a nucleic acid sequence is provided which encodes for the GLP-1 peptides described herein. In some embodiments, this may include any nucleic acid sequence which encodes the GLP-1 sequence of SEQ ID NO: 1. In another embodiment, this includes any nucleic acid which includes the GLP-1 sequence of SEQ ID NO: 2. In another embodiment, this includes any nucleic acid which includes the GLP-1 sequence of SEQ ID NO: 3. In another embodiment, this includes any nucleic acid which includes the GLP-1 sequence of SEQ ID NO: 4. In another embodiment, this includes any nucleic acid which includes the GLP-1 sequence of SEQ ID NO: 5. In another embodiment, this includes any nucleic acid which includes the GLP-1 sequence of SEQ ID NO: 6.

In one embodiment, a nucleic acid sequence is provided which encodes for the GLP-1 fusion protein described herein. In another embodiment, this includes any nucleic acid sequence which encodes the GLP-1 fusion protein of SEQ ID NO: 14.

### Expression Cassettes

Provided herein, in another aspect, is an expression cassette comprising a nucleic acid encoding a GLP-1 fusion protein as described herein. As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences (also referred to as elements) that are contiguous or non-contiguous with the nucleic acid sequence and regulatory sequences that act in trans or cis nucleic acid sequence. Such regulatory sequences typically include, e.g., one or more of a promoter, an enhancer, a transcription factor, transcription terminator, an intron, sequences that enhance translation efficiency (i.e., a Kozak consensus sequence), efficient RNA processing signals such as slicing and a polyadenylation sequence, sequences that stabilize cytoplasmic mRNA, for example Woodchuck Hepatitis Virus (WHP) posttranslational Regulatory Element (WPRE), and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

In one embodiment, the expression cassette refers to a nucleic acid molecule which comprises the GLP-1 construct coding sequences (e.g., coding sequences for the GLP-1 fusion protein), promoter, and may include other regulatory sequences therefor, which cassette may be engineered into a genetic element and/or packaged into the capsid of a viral vector (e.g., a viral particle). Typically, such an expression cassette for generating a viral vector contains the GLP-1 construct sequences described herein flanked by packaging signals of the viral genome (and is termed a “vector genome”) and other expression control sequences such as those described herein. Any of the expression control sequences can be optimized for a specific species using techniques known in the art including, e.g., codon optimization, as described herein.

In certain embodiments, the expression cassette includes a constitutive promoter. In another embodiment, a CB7 promoter is used. CB7 is a chicken β-actin promoter with cytomegalovirus enhancer elements. In some embodiments, the CB7 promoter has the nucleic acid sequence of SEQ ID NO: 33. In one embodiment, the promoter is a CMV promoter. In some embodiments, the CMV promoter is a nucleic acid sequence of SEQ ID NO: 27.

In another embodiment, a tissue specific promoter is used. Alternatively, other liver-specific promoters may be used such as those listed in the Liver Specific Gene Promoter Database, Cold Spring Harbor, (rulai.schl.edu/LSPD), and including, but not limited to, alpha 1 anti-trypsin (A1AT); human albumin (Miyatake et al., J. Virol., 71:5124 32 (1997)), humAlb; hepatitis B virus core promoter (Sandig et al., Gene Ther., 3:1002 9 (1996)); a TTR minimal enhancer/promoter, alpha-antitrypsin promoter, liver-specific promoter (LSP) (Wu et al. Mol Ther. 16:280-289 (2008)), TBG liver specific promoter. Other promoters, such as viral promoters, constitutive promoters, regulatable promoters (see, e.g., WO 2011/126808 and WO 2013/04943), or a promoter responsive to physiologic cues may be used may be utilized in the vectors described herein.

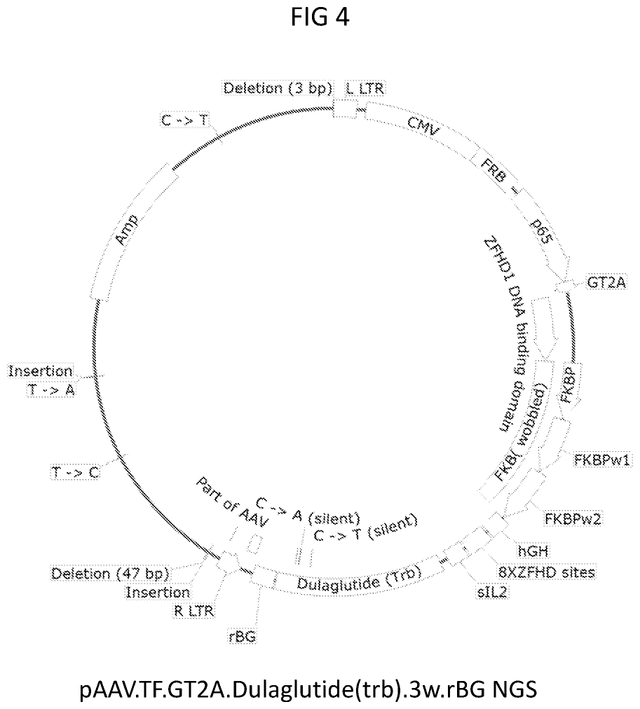

In one embodiment, the promoter is comprised in an inducible gene expression system. The inducible gene regulation/expression system contains at least the following components: a promoter operably linked to transgene encoding the GLP-1 fusion protein described herein (also referred to as the regulatable promoter), an activation domain, DNA binding domain, and zinc finger homeodomain binding site(s). In other embodiments, additional components may be included in the expression system, as further described herein. A plasmid showing design of an exemplary inducible expression system is shown in FIG. 4.

The system comprises the promoter upstream of the coding sequence for the GLP-1 fusion protein. Promoters described herein, such as CMV and CB7 promoters may be used. In one embodiment, the promoter is a CMV promoter, such as that shown in SEQ ID NO: 27. In another embodiment, the promoter is the ubiquitous, inducible promoter Z12I which comprises 12 repeated copies of the binding site for ZFHD1 and the IL2 minimal promoter. See, e.g., Chen et al, Hum Gene Ther Methods. 2013 August; 24(4): 270-278, which is incorporated herein.

The expression system comprises an activation domain, which is preferably located upstream of the DNA binding domain. In one embodiment, the activation domain is a fusion of the carboxy terminus from the p65 subunit of NF-kappa B and FKBP12-rapamycin binding (FRB) domain of FKBP12-rapamycin-associated protein (FRAP). In one embodiment, the activation domain is a FKBP12-rapamycin binding (FRB) domain of human FKBP12-rapamycin-associated protein (FRAP) fused to a carboxy terminus from the p65 subunit of NF-kappa B from a human. In one embodiment, the FRB domain has the amino acid sequence shown in SEQ ID NO: 24. In one embodiment, the FRB domain has the amino acid sequence shown in SEQ ID NO: 24 encoded by nucleic acid sequence of SEQ ID NO: 23. In one embodiment, the p65 subunit has the sequence shown in SEQ ID NO: 26. In one embodiment, the p65 subunit has the sequence shown in SEQ ID NO: 26 encoded by nucleic acid sequence of SEQ ID NO: 25.

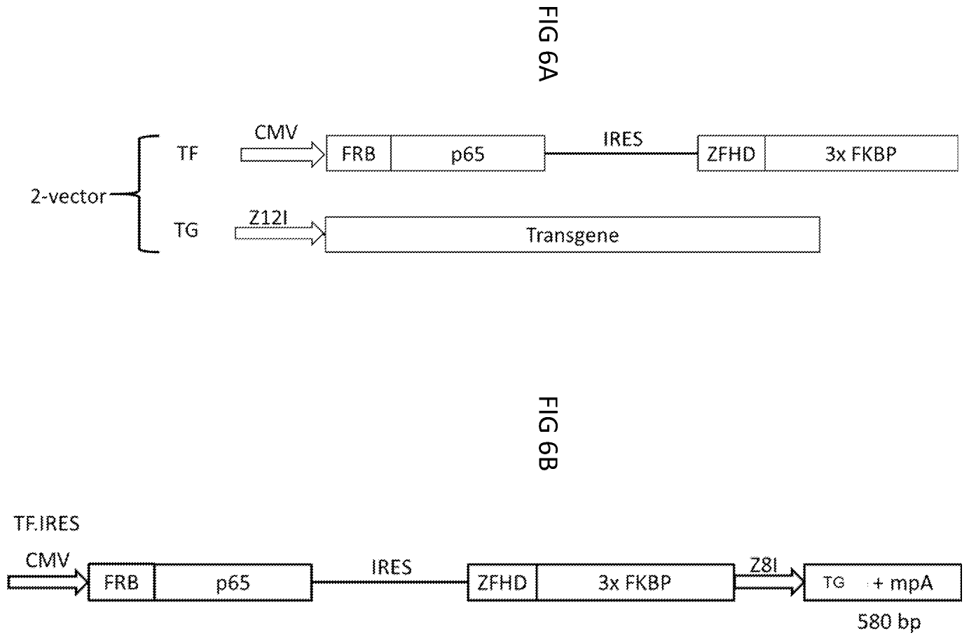

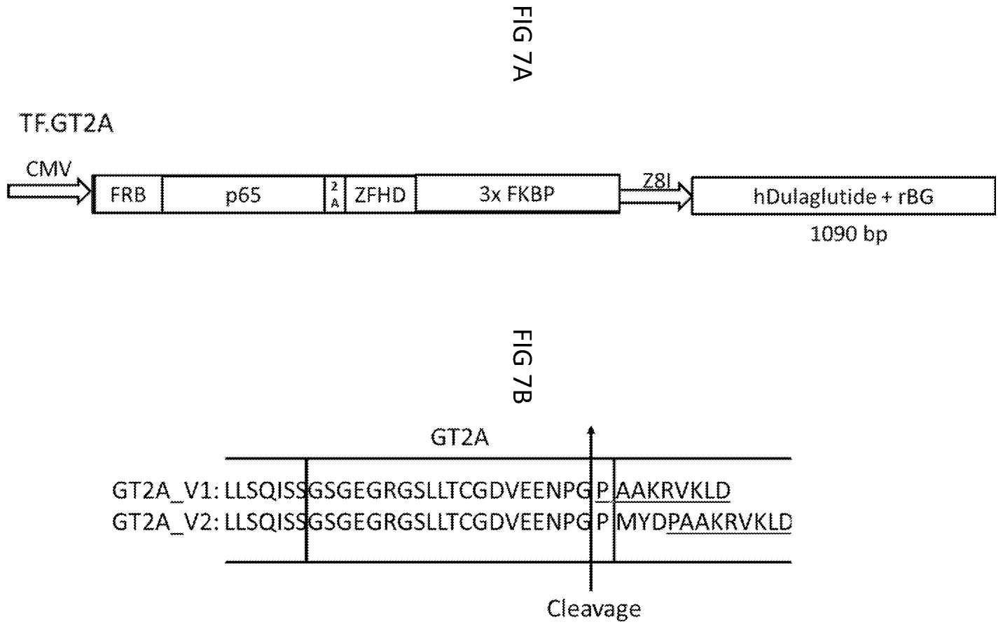

The inducible system may be comprised in a single vector that comprises the coding sequence for the fusion protein, or in a two-vector system. Examples of a 2-vector (FIG. 6A) and 1-vector (FIG. 6B and FIG. 7A) systems incorporating GLP1 fusion proteins are described herein.

In one embodiment, there is a linker between the transactivation domain and DNA binding domain, which linker may be an F2A or an IRES. In one embodiment the linker is selected from an IRES or a 2A peptide. In one embodiment, the linker is a cleavable 2A peptide. In one embodiment, the linker comprises a GT2A_V1 peptide comprising an amino acid sequence of SEQ ID NO: 21. In one embodiment, the linker comprises a GT2A_V2 peptide comprising an amino acid sequence of SEQ ID NO: 22. In one embodiment, the 2A peptide is selected to increase the packaging limit to allow for a single vector system.

The DNA binding domain is composed of a DNA-binding fusion of zinc finger homeodomain 1 (ZFHD1) joined to up to three copies of FK506 binding protein (FKBP). In the presence of an inducing agent, e.g., a rapalog such as rapamycin, the DNA binding domain and activation domain are dimerized through interaction of their FKBP and FRB domains, leading to transcription activation of the transgene. In some embodiments, the ZFHD1 is included in frame with the GT2A or IRES. In one embodiment, the ZFHD1 has the sequence shown in SEQ ID NO: 29. In one embodiment, the ZFHD1 has the sequence of SEQ ID NO: 28 encoded by a nucleic acid sequence of SEQ ID NO: 28.

The expression system is designed to have one, two or three copies of the FKBP sequence. These are termed herein FKBP subunits. In one embodiment, the subunits are designed to express the same protein, but to have nucleic acids which are divergent from one another in order to minimize recombination. For example, SEQ ID NO: 30 provides 3 “wobbled” coding sequences for FKBP, each of which encode the sequence shown in SEQ ID NO: 31:

GVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLV
FDVELLKLE

The expression system further comprises zinc finger homeodomain binding sites. The nucleic acid molecule contains at least 1, 2, 3, 4, 5, 6, 7, 8, 9, 10, 11 or 12 binding sites for ZFHD. In one embodiment, the expression system contains 8 (eight) zinc finger homeodomains binding site (binding partners) (8XZFHD). However, the invention encompasses expression systems having from two to about twelve copies of the zinc finger binding site. An example of a single copy of a ZFHD binding site is: aatgatgggcgctcgagt (SEQ ID NO: 32)

In some embodiments, there is a minimal IL2 promoter downstream of the zinc finger homeodomain binding sites. An exemplary IL2 promoter is shown in SEQ ID NO: 10.

Such inducible systems are known in the art, and include, e.g., the rapamycin-inducible system described by e.g., Rivera et al, A humanized system for pharmacologic control of gene expression, Nature Medicine volume 2, pages 1028-1032 (September 1996) and Rivera et al, Long-term pharmacologically regulated expression of erythropoietin in primates following AAV-mediated gene transfer, Blood, 15 Feb. 2005, volume 105, number 4, both of which are incorporated herein by reference. In one embodiment, the inducible gene expression system comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A peptide, ZFHD1 DNA binding domain, three FKBP subunits, an hGH poly A, 8XZFHD, and a minimal sIL2 promoter. These sequences are in addition to the coding sequence for the GLP-1 fusion protein and optionally other regulatory sequences.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), human growth hormone (hGH), SV40, rabbit β-globin (also referred to as rabbit globin polyA; RGB), modified RGB (mRGB) and TK polyA. Examples of suitable enhancers include, e.g., the alpha fetoprotein enhancer, the TTR minimal promoter/enhancer, LSP (TH-binding globulin promoter/alphal-microglobulin/bikunin enhancer), amongst others. In one embodiment, the polyA is a rabbit globin polyA.

These control sequences are “operably linked” to the GLP-1 construct sequences. As used herein, the term “operably linked” refers to both expression control sequences that are contiguous with the gene of interest and expression control sequences that act in trans or at a distance to control the gene of interest.

In one embodiment, a rAAV is provided which includes a 5′ ITR, CB7 promoter, chicken beta-actin intron, coding sequence for the fusion protein of SEQ ID NO: 14, a rabbit globin poly A, and a 3′ ITR. In another embodiment, the rAAV comprises a polynucleotide comprising a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A peptide, ZFHD1 DNA binding domain, three FKBP subunits, an hGH poly A, 8XZFHD, a minimal sIL2 promoter, coding sequence for the GLP-1 fusion protein of SEQ ID NO: 14, and rabbit beta globin polyA.

In one embodiment, an expression cassette is provided that includes a polynucleotide comprising a CB7 promoter, chicken beta-actin intron, coding sequence for the fusion protein of SEQ ID NO: 14, and a rabbit globin poly A. In one embodiment, the expression cassette is that found in SEQ ID NO: 34, or a sequence sharing at least 70%, 75%, 80%, 85%, 90%, 95%, 99% or 100% identity therewith. In another embodiment, a vector genome is provided wherein SEQ ID NO: 34, or a sequence sharing at least 70%, 75%, 80%, 85%, 90%, 95%, 99% or 100% identity therewith is flanked by 5′ and 3′ AAV ITRs.

In another embodiment, an expression cassette is provided that includes a polynucleotide comprising a CB7 promoter, chicken beta-actin intron, coding sequence for the fusion protein of SEQ ID NO: 37, and a rabbit globin poly A. In one embodiment, the expression cassette is that found in SEQ ID NO: 35, or a sequence sharing at least 70%, 75%, 80%, 85%, 90%, 95%, 99% or 100% identity therewith. In another embodiment, a vector genome is provided wherein SEQ ID NO: 35, or a sequence sharing at least 70%, 75%, 80%, 85%, 90%, 95%, 99% or 100% identity therewith is flanked by 5′ and 3′ AAV ITRs.

In another embodiment, an expression cassette is provided that includes a polynucleotide comprising a CMV promoter, a FKBP12-rapamycin binding (FRB) domain of human FKBP12-rapamycin-associated protein (FRAP) fused to a carboxy terminus from the p65 subunit of NF-kappa B from a human, GT2A peptide, ZFHD1 DNA binding domain, three FKBP subunits, 8XZFHD, a minimal IL2 promoter, coding sequence for the GLP-1 fusion protein of SEQ ID NO: 14, and rabbit beta globin polyA. In one embodiment, the expression cassette is that found in SEQ ID NO: 38, or a sequence sharing at least 70%, 75%, 80%, 85%, 90%, 95%, 99% or 100% identity therewith. In another embodiment, a vector genome is provided wherein SEQ ID NO: 38, or a sequence sharing at least 70%, 75%, 80%, 85%, 90%, 95%, 99% or 100% identity therewith is flanked by 5′ and 3′ AAV ITRs.

In another embodiment, an expression cassette is provided that includes a polynucleotide comprising a CMV promoter, a FKBP12-rapamycin binding (FRB) domain of human or rhesus FKBP12-rapamycin-associated protein (FRAP) fused to a carboxy terminus from the p65 subunit of NF-kappa B from a human or rhesus, GT2A peptide, ZFHD1 DNA binding domain, three FKBP subunits, 8XZFHD, a minimal IL2 promoter, coding sequence for the GLP-1 fusion protein of SEQ ID NO: 37, and rabbit beta globin polyA. In one embodiment, the expression cassette is that found in SEQ ID NO: 39, or a sequence sharing at least 70%, 75%, 80%, 85%, 90%, 95%, 99% or 100% identity therewith. In another embodiment, a vector genome is provided wherein SEQ ID NO: 39, or a sequence sharing at least 70%, 75%, 80%, 85%, 90%, 95%, 99% or 100% identity therewith is flanked by 5′ and 3′ AAV ITRs.

In another embodiment, an expression cassette is provided that includes a polynucleotide comprising a Z12I promoter (comprising 12 ZFHD1 sites and a minimal IL2 promoter), coding sequence for the GLP-1 fusion protein of SEQ ID NO: 37, and rabbit beta globin polyA. In one embodiment, the expression cassette is that found in SEQ ID NO: 40, or a sequence sharing at least 70%, 75%, 80%, 85%, 90%, 95%, 99% or 100% identity therewith. In another embodiment, a vector genome is provided wherein SEQ ID NO: 40, or a sequence sharing at least 70%, 75%, 80%, 85%, 90%, 95%, 99% or 100% identity therewith is flanked by 5′ and 3′ AAV ITRs. A second expression cassette is provided that includes a polynucleotide comprising a CMV promoter, a chimeric intron, a FKBP12-rapamycin binding (FRB) domain of human or rhesus FKBP12-rapamycin-associated protein (FRAP) fused to a p65 subunit of NF-kappa B from a human or rhesus (or a portion thereof), an IRES or 2A peptide, ZFHD1 DNA binding domain, three FKBP subunits, 8XZFHD, and a polyA sequence. In one embodiment, the expression cassette is that found in SEQ ID NO: 41, or a sequence sharing at least 70%, 75%, 80%, 85%, 90%, 95%, 99% or 100% identity therewith. In another embodiment, a vector genome is provided wherein SEQ ID NO: 41, or a sequence sharing at least 70%, 75%, 80%, 85%, 90%, 95%, 99% or 100% identity therewith is flanked by 5′ and 3′ AAV ITRs.

### Viral Vectors

In another aspect, viral vectors that include the expression cassettes described herein are provided. In certain embodiments of the viral vectors described herein, the viral vector is an adeno-associated virus (AAV) viral vector or recombinant AAV (rAAV). The term “recombinant AAV” or “rAAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged an expression cassette flanked by AAV inverted terminal repeat sequences (ITRs) (together referred to as the “vector genome”) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAVrh91 capsid or variant thereof. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAVAnc80, AAV10, AAV11, AAV12, AAVrh8, AAVrh74, AAV-DJ8, AAV-DJ, AAVhu.37, AAVrh.64R1, and AAVhu68. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10), WO 2005/033321, WO 2018/160582 (AAVhu68), which are incorporated herein by reference. Other suitable AAVs may include, without limitation, AAVrh90 [PCT/US20/30273, filed Apr. 28, 2020], AAVrh91 [PCT/US20/030266, filed Apr. 28, 2020, now a publication WO 2020/223231, published Nov. 5, 2020], AAVrh92, AAVrh93, AAVrh91.93 [PCT/US20/30281, filed Apr. 28, 2020], which are incorporated by reference herein. Other suitable AAV include AAV3B variants which are described in U.S. Provisional Patent Application No. 62/924,112, filed Oct. 21, 2019, and U.S. Provisional Patent Application No. 63/025,753, filed May 15, 2020, describing AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. See also, International Patent Application No. PCT/US21/45945, filed Aug. 13, 2021, U.S. Provisional Patent Application No. 63/065,616, filed Aug. 14, 2020, and U.S. Provisional Patent Application No. 63/109,734, filed Nov. 4, 2020, which are all incorporated herein by reference in its entireties. These documents also describe other AAV capsids which may be selected for generating rAAV and are incorporated by reference. Among the AAVs isolated or engineered from human or nonhuman primates (NHP) and well characterized, human AAV2 is the first AAV that was developed as a gene transfer vector; it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

In one embodiment, the viral vector is an rAAV having the capsid of AAV8 or a functional variant thereof. In one embodiment, the viral vector is an rAAV having the capsid of AAVrh91 or a functional variant thereof. In one embodiment, the viral vector is an rAAV having the capsid of AAV3.AR.2.12 or a functional variant thereof. In one embodiment, the viral vector is an rAAV having a capsid selected from AAV9, AAVrh64R1, AAVhu37, or AAVrh10.

In certain embodiments, a novel isolated AAVrh91 capsid is provided. A nucleic acid sequence encoding the AAVrh91 capsid is provided in SEQ ID NO: 18 and the encoded amino acid sequence is provided in SEQ ID NO: 20. Provided herein is an rAAV comprising at least one of the vp1, vp2 and the vp3 of AAVrh91 (SEQ ID NO: 20). Also provided herein are rAAV comprising an AAV capsid encoded by at least one of the vp1, vp2 and the vp3 of AAVrh91 (SEQ ID NO: 18). In yet another embodiment, a nucleic acid sequence encoding the AAVrh91 amino acid sequence is provided in SEQ ID NO: 19 and the encoded amino acid sequence is provided in SEQ ID NO: 20. Also provided herein are rAAV comprising an AAV capsid encoded by at least one of the vp1, vp2 and the vp3 of AAVrh91eng (SEQ ID NO: 19). In certain embodiments, the vp1, vp2 and/or vp3 is the full-length capsid protein of AAVrh91 (SEQ ID NO: 20). In other embodiments, the vp1, vp2 and/or vp3 has an N-terminal and/or a C-terminal truncation (e.g., truncation(s) of about 1 to about 10 amino acids).

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 20, vp1 proteins produced from SEQ ID NO: 18, or vp1 proteins produced from a nucleic acid sequence at least 70% identical to SEQ ID NO: 18 which encodes the predicted amino acid sequence of 1 to 736 of SEQ ID NO: 20, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 20, vp2 proteins produced from a sequence comprising at least nucleotides 412 to 2208 of SEQ ID NO: 18, or vp2 proteins produced from a nucleic acid sequence at least 70% identical to at least nucleotides 412 to 2208 of SEQ ID NO: 18 which encodes the predicted amino acid sequence of at least about amino acids 138 to 736 of SEQ ID NO: 20, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 20, vp3 proteins produced from a sequence comprising at least nucleotides 607 to 2208 of SEQ ID NO: 18, or vp3 proteins produced from a nucleic acid sequence at least 70% identical to at least nucleotides 607 to 2208 of SEQ ID NO: 18 which encodes the predicted amino acid sequence of at least about amino acids 203 to 736 of SEQ ID NO: 20; and/or (2) a heterogeneous population of vp1 proteins which are the product of a nucleic acid sequence encoding the amino acid sequence of SEQ ID NO: 20, a heterogeneous population of vp2 proteins which are the product of a nucleic acid sequence encoding the amino acid sequence of at least about amino acids 138 to 736 of SEQ ID NO: 20, and a heterogeneous population of vp3 proteins which are the product of a nucleic acid sequence encoding at least amino acids 203 to 736 of SEQ ID NO: 20, wherein: the vp1, vp2 and vp3 proteins contain subpopulations with amino acid modifications comprising at least two highly deamidated asparagines (N) in asparagine-glycine pairs in SEQ ID NO: 20 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAVrh91 capsid is characterized by one or more of the following: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 20, vp1 proteins produced from SEQ ID NO: 19, or vp1 proteins produced from a nucleic acid sequence at least 70% identical to SEQ ID NO: 19 which encodes the predicted amino acid sequence of 1 to 736 of SEQ ID NO: 20, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 20, vp2 proteins produced from a sequence comprising at least nucleotides 412 to 2208 of SEQ ID NO: 19, or vp2 proteins produced from a nucleic acid sequence at least 70% identical to at least nucleotides 412 to 2208 of SEQ ID NO: 19 which encodes the predicted amino acid sequence of at least about amino acids 138 to 736 of SEQ ID NO: 20, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 20, vp3 proteins produced from a sequence comprising at least nucleotides 607 to 2208 of SEQ ID NO: 19, or vp3 proteins produced from a nucleic acid sequence at least 70% identical to at least nucleotides 607 to 2208 of SEQ ID NO: 19 which encodes the predicted amino acid sequence of at least about amino acids 203 to 736 of SEQ ID NO: 20; and/or (2) a heterogeneous population of vp1 proteins which are the product of a nucleic acid sequence encoding the amino acid sequence of SEQ ID NO: 20, a heterogeneous population of vp2 proteins which are the product of a nucleic acid sequence encoding the amino acid sequence of at least about amino acids 138 to 736 of SEQ ID NO: 20, and a heterogeneous population of vp3 proteins which are the product of a nucleic acid sequence encoding at least amino acids 203 to 736 of SEQ ID NO: 20, wherein: the vp1, vp2 and vp3 proteins contain subpopulations with amino acid modifications comprising at least two highly deamidated asparagines (N) in asparagine-glycine pairs in SEQ ID NO: 20 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the AAVrh91 vp1, vp2 and vp3 proteins contain subpopulations with amino acid modifications comprising at least two highly deamidated asparagines (N) in asparagine-glycine pairs in SEQ ID NO: 20 and optionally further comprising subpopulations comprising other deamidated amino acids, wherein the deamidation results in an amino acid change. High levels of deamidation at N-G pairs N57, N383 and/or N512 are observed, relative to the number of SEQ ID NO: 20. Deamidation has been observed in other residues. In certain embodiments, AAVrh91 may have other residues deamidated, e.g., typically at less than 10% and/or may have other modifications, including phosphorylation (e.g., where present, in the range of about 2 to about 30%, or about 2 to about 20%, or about 2 to about 10%) (e.g., at S149), or oxidation (e.g, at one or more of ˜W22, ˜M211, W247, M403, M435, M471, W478, W503, ˜M537, ˜M541, ˜M559, ˜M599, M635, and/or, W695). Optionally the W may oxidize to kynurenine.

TABLE A
AAVrh91 Deamidation

| AAVrh91 | |
|---|---|
| Deamidation based | |
| on VP1 numbering | % Deamidation |
| N57 + Deamidation | 65-90, 70-95, 80-95, 75-100, 80-100, or 90-100 |
| N94 + Deamidation | 2-15 or 2-5 |
| N303 + Deamidation | 2-15 or 5-10 |
| N383 + Deamidation | 65-90, 70-95, 80-95, 75-100, 80-100, or 90-100 |
| N497 + Deamidation | 2-15 or 5-10 |
| N512 + Deamidation | 65-90, 70-95, 80-95, 75-100, 80-100, or 90-100 |
| ~N691 + Deamidation | 2-15, 2-10, or 5-10 |

In certain embodiments, an AAVrh91 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh91 sequence provided herein. See, SEQ ID NO: 20.

In certain embodiments, an AAVrh91 capsid comprises: a heterogeneous population of vp1 proteins which are the product of a nucleic acid sequence encoding the amino acid sequence of SEQ ID NO: 20, a heterogeneous population of vp2 proteins which are the product of a nucleic acid sequence encoding the amino acid sequence of at least about amino acids 138 to 736 of SEQ ID NO: 20, and a heterogeneous population of vp3 proteins which are the product of a nucleic acid sequence encoding at least amino acids 203 to 736 of SEQ ID NO: 20.

In certain embodiments, the modified AAVrh91 nucleic acid sequences is be used to generate a mutant rAAV having a capsid with lower deamidation than the native AAVrh91 capsid. Such mutant rAAV may have reduced immunogenicity and/or increase stability on storage, particularly storage in suspension form.

In one aspect, a recombinant AAV (rAAV) is provided. The rAAV includes an AAV capsid from adeno-associated virus rh91, and a vector genome packaged in the AAV capsid, said vector genome comprising AAV inverted terminal repeats (ITRs), a coding sequence for the GLP-1 receptor agonist of SEQ ID NO: 14, and regulatory sequences which direct expression of the GLP-1 receptor agonist.

In one embodiment, the rAAV is an scAAV. The abbreviation “sc” refers to self-complementary. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In one embodiment, the nucleic acid sequences encoding the GLP-1 constructs described herein are engineered into any suitable genetic element, e.g., naked DNA, phage, transposon, cosmid, RNA molecule (e.g., mRNA), episome, etc., which transfers the GLP-1 sequences carried thereon to a host cell, e.g., for generating nanoparticles carrying DNA or RNA, viral vectors in a packaging host cell and/or for delivery to a host cell in a subject. In one embodiment, the genetic element is a plasmid. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, the term “host cell” may refer to the packaging cell line in which a vector (e.g., a recombinant AAV or rAAV) is produced from a production plasmid. In the alternative, the term “host cell” may refer to any target cell in which expression of a gene product described herein is desired. Thus, a “host cell,” refers to a prokaryotic or eukaryotic cell (e.g., bacterial cell, human cell or insect cell) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. In a further embodiment, the term “host cell” is an intestine cell, a small intestine cell, a pancreatic cell, a liver cell.

As used herein, the term “target cell” refers to any target cell in which expression of a heterologous nucleic acid sequence or protein is desired. In certain embodiments, the target cell is a liver cell. In other embodiments, the target cell is a muscle cell.

In one embodiment, the rAAV is provided which comprises a vector genome comprising an expression cassette, wherein the expression cassette comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A_V1 peptide, ZFHD1 DNA binding domain, three FKBP subunits, an hGH poly A, 8XZFHD, a minimal sIL2 promoter, coding sequence for the GLP-1 fusion protein of SEQ ID NO: 14, and rabbit beta globin polyA. In another embodiment, the rAAV is provide which comprises a vector genome comprising an expression cassette, wherein the expression cassette comprises a CMV promoter, the activation domain is a FKBP12-rapamycin binding (FRB) domain of human FKBP12-rapamycin-associated protein (FRAP) fused to a carboxy terminus from the p65 subunit of NF-kappa B from a human, GT2A_V2 peptide, ZFHD1 DNA binding domain, three FKBP subunits, an hGH poly A, 8XZFHD, a minimal sIL2 promoter, coding sequence for the GLP-1 fusion protein of SEQ ID NO: 14, and rabbit beta globin polyA.

The minimal sequences required to package the expression cassette into an AAV viral particle are the AAV 5′ and 3′ ITRs, which may be of the same AAV origin as the capsid, or of a different AAV origin (to produce an AAV pseudotype). In one embodiment, the ITR sequences from AAV2, or the deleted version thereof (AITR), are used for convenience and to accelerate regulatory approval. However, ITRs from other AAV sources may be selected. Preferably, the source of the ITRs is the same as the source of the Rep protein, which is provided in trans for production. Typically, an expression cassette for an AAV vector comprises an AAV 5′ ITR, the GLP-1 fusion protein coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used.

For packaging an expression cassette into virions, the ITRs are the only AAV components required in cis in the same construct as the gene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. In one embodiment, a chimeric AAV capsid may be utilized. Still other AAV components may be selected. Sources of such AAV sequences are described herein and may also be isolated or obtained from academic, commercial, or public sources (e.g., the American Type Culture Collection, Manassas, VA). The AAV sequences may be obtained through synthetic or other suitable means by reference to published sequences such as are available in the literature or in databases such as, e.g., GenBank®, PubMed®, or the like.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus ULS, ULB, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

The rAAV described herein comprise a selected capsid with a vector genome packaged inside. The vector genome (or rAAV genome) comprises 5′ and 3′ AAV inverted terminal repeats (ITRs), the polynucleotide sequence encoding the fusion protein, and regulatory sequences which direct insertion of the polynucleotide sequence encoding the fusion protein to the genome of a host cell. In one embodiment, the vector genome is the sequence shown in SEQ ID NO: 16 or a sequence sharing at least 70%, at least 75%, at least 80%, at least 85%, at least 90%, at least 95%, or at least 99% identity therewith.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding GLP-1 constructs operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences from AAV2. However, ITRs from other AAV sources may be selected. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type length of 145 base pairs during vector DNA amplification using the internal (A′) element as a template. In other embodiments, full-length AAV 5′ and 3′ ITRs are used. Where the source of the ITRs is from AAV2 and the AAV capsid is from another AAV source, the resulting vector may be termed pseudotyped. However, other configurations of these elements may be suitable.

Optionally, the GLP-1 constructs described herein may be delivered via viral vectors other than rAAV. Such other viral vectors may include any virus suitable for gene therapy, including but not limited to adenovirus; herpes virus; lentivirus; retrovirus; etc. Suitably, where one of these other vectors is generated, it is produced as a replication-defective viral vector.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Also provided are compositions which include the viral vector constructs described herein. The pharmaceutical compositions described herein are designed for delivery to subjects in need thereof by any suitable route or a combination of different routes. Direct delivery to the liver (optionally via intravenous, via the hepatic artery, or by transplant), oral, inhalation, intranasal, intratracheal, intraarterial, intraocular, intravenous, intramuscular, subcutaneous, intradermal, and other parental routes of administration. The viral vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus). In one embodiment, administration is intramuscular. In another embodiment, administration is intravenous.

The replication-defective viruses can be formulated with a physiologically acceptable carrier for use in gene transfer and gene therapy applications. In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The nuclease resistant particles are then subjected to heat treatment to release the genome from the capsid. The released genomes are then quantitated by real-time PCR using primer/probe sets targeting specific region of the viral genome (usually poly A signal). Another suitable method for determining genome copies are the quantitative-PCR (qPCR), particularly the optimized qPCR or digital droplet PCR [Lock Martin, et al, Human Gene Therapy Methods. April 2014, 25(2): 115-125. doi:10.1089/hgtb.2013.131, published online ahead of editing Dec. 13, 2013].

Also, the replication-defective virus compositions can be formulated in dosage units to contain an amount of replication-defective virus that is in the range of about 1.0×10⁹ GC to about 1.0×10¹⁵ GC. In another embodiment, this amount of viral genome may be delivered in split doses. In one embodiment, the dose is about 1.0×10¹⁰ GC to about 3.0×10¹⁴ GC for an average human subject of about 70 kg. In another embodiment, the dose about 1×10⁹ GC. For example, the dose of AAV virus may be about 1×10¹⁰ GC, 1×10¹¹ GC, about 5×10¹¹ GC, about 1×10¹² GC, about 5×10¹² GC, or about 1×10¹¹ GC. In another embodiment, the dosage is about 1.0×10⁹ GC/kg to about 3.0×10¹⁴ GC/kg for a human subject. In another embodiment, the dose about 1×10⁹ GC/kg. For example, the dose of AAV virus may be about 1×10¹⁰ GC/kg, 1×10¹¹ GC/kg, about 5×10¹¹ GC/kg, about 1×10¹² GC/kg, about 5×10¹² GC/kg, or about 1×10¹³ GC/kg. In one embodiment, the constructs may be delivered in volumes from 1 μL to about 100 mL. As used herein, the term “dosage” or “amount” can refer to the total dosage or amount delivered to the subject in the course of treatment, or the dosage or amount delivered in a single unit (or multiple unit or split dosage) administration.

The above-described recombinant vectors may be delivered to host cells according to published methods. The rAAV, preferably suspended in a physiologically compatible carrier, may be administered to a desired subject including a human. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

In another embodiment, the composition includes a carrier, diluent, excipient and/or adjuvant. In certain embodiments, for administration to a human patient, the rAAV is suitably suspended in an aqueous solution containing saline, a surfactant, and a pharmaceutically and/or physiologically compatible salt or mixture of salts. Suitably, the formulation is adjusted to a physiologically acceptable pH, e.g., in the range of pH 6 to 9, or pH 6.0 to 7.5, or pH 6.2 to 7.7, or pH 6.5 to 7.5, pH 7.0 to 7.7, or pH 7.2 to 7.8, or about 7.0. In certain embodiments, the formulation is adjusted to a pH of about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8. In certain embodiments, a pH of about 7.28 to about 7.32, about 6.0 to about 7.5, about 6.2 to about 7.7, about 7.5 to about 7.8, about 6.0, about 6.1, about 6.2, about 6.3, about 6.4, about 6.5, about 6.6, about 6.7, about 6.8, about 6.9, about 7.0, about 7.1, about 7.2, about 7.3 about 7.4, about 7.5, about 7.6, about 7.7, or about 7.8 may be desired. In certain embodiments, for intravenous delivery, a pH of about 6.8 to about 7.2 may be desired. However, other pHs within the broadest ranges and these subranges may be selected for other route of delivery.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

Dosages of the vector depends primarily on factors such as the condition being treated, the age, weight and health of the patient, and may thus vary among patients. For example, a therapeutically effective human dosage of viral vector is generally in the range of from about 25 to about 1000 microliters to about 100 mL of solution containing concentrations of from about 1×10⁹ to 1×10¹⁶ genomes virus vector (to treat an average subject of 70 kg in body weight) including all integers or fractional amounts within the range, and preferably 1.0×10¹² GC to 1.0×10¹³ GC for a human patient. The composition of the invention may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

In some embodiments, a concentration of a recombinant adeno-associated virus carrying a nucleic acid sequence encoding the desired transgene under the control of the regulatory sequences desirably ranges from about 10⁷ and 10¹⁴ vector genomes per milliliter (vg/mL) (also called genome copies/mL (GC/mL)) in a composition.

In one embodiment, the dosage of rAAV in a composition is from about 1.0×10⁹ GC/kg of body weight to about 1.5×10¹³ GC/kg. In one embodiment, the dosage is about 1.0×10¹⁰ GC/kg. In one embodiment, the dosage is about 1.0×10¹¹ GC/kg. In one embodiment, the dosage is about 1.0×10¹² GC/kg. In one embodiment, the dosage is about 5.0×10¹² GC/kg. In one embodiment, the dosage is about 1.0×10¹³ GC/kg. All ranges described herein are inclusive of the endpoints.

In one embodiment, the effective dosage (total genome copies delivered) is from about 10⁷ to 10¹³ vector genomes. In one embodiment, the total dosage is about 108 genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed.

In certain embodiments, the composition comprises an rAAV comprising an inducible GLP-1 agonist construct. In certain embodiments, the inducing agent or molecule is a rapamycin or a rapalog. In certain embodiments, the inducing agent is rapamycin, and is administered at least one or more, at least two or more, at least three or more times following rAAV-comprising composition. In some embodiments the rapamycin is administered at dose at least about 4 to at least about 40 nM. In certain embodiments, the inducing agent (i.e., rapamycin) is administered at a dose at least about 0.1 mg/kg to at least about 3.0 mg/kg. In certain embodiments, the inducing agent (i.e., rapamycin) is administered at a dose at least about 0.5 mg/kg to at least about 2.0 mg/kg.

The viral vectors and other constructs described herein may be used in preparing a medicament for delivering a GLP-1 fusion protein construct to a subject in need thereof, supplying GLP-1 having an increased half-life to a subject, and/or for treating type I diabetes, type II diabetes or metabolic syndrome in a subject. Thus, in another aspect a method of treating diabetes is provided. The method includes administering a composition as described herein to a subject in need thereof. In one embodiment, the composition includes a viral vector containing a GLP-1 fusion protein expression cassette, as described herein.

As used herein, the term “treatment” or “treating” is defined encompassing administering to a subject one or more compounds or compositions described herein for the purposes of amelioration of one or more symptoms of type I diabetes, type II diabetes or metabolic syndrome. “Treatment” can thus include one or more of reducing progression of type I diabetes, type II diabetes or metabolic syndrome, reducing the severity of the symptoms, removing the disease symptoms, delaying progression of disease, or increasing efficacy of therapy in a given subject.

As used herein, the term “remission” refers to the ability to cease insulin treatment when the subject no longer exhibits clinical signs of diabetes and has normal blood glucose levels.

In another embodiment, a method for treating T2DM in a subject is provided. The method includes administering a viral vector comprising a nucleic acid molecule comprising a sequence encoding a fusion protein as described herein. In one embodiment, the subject is a human.

In another aspect, a method of treating a metabolic disease in a subject is provided. The method includes administering a composition as described herein to a subject in need thereof. In one embodiment, the composition includes a viral vector containing a GLP-1 fusion protein expression cassette, as described herein. In one embodiment, the metabolic disease is Type I diabetes. In one embodiment, the metabolic disease is Type II diabetes. In one embodiment, the metabolic disease is metabolic syndrome. In one embodiment the subject is a human.

In another aspect a method of reducing body weight in a subject is provided. The method includes administering a composition as described herein to a subject in need thereof. In one embodiment, the composition includes a viral vector containing a GLP-1 fusion protein expression cassette, as described herein.

A course of treatment may optionally involve repeat administration of the same viral vector (e.g., an AAVrh91 vector) or a different viral vector (e.g., an AAVrh91 and an AAV3B.AR2.12). Still other combinations may be selected using the viral vectors described herein. Optionally, the composition described herein may be combined in a regimen involving other diabetic drugs or protein-based therapies (including e.g., GLP-1 analogues, insulin, oral antihyperglycemic drugs (sulfonylureas, biguanides, thiazolidinediones, and alpha-glucoidase inhibitors). Optionally, the composition described herein may be combined in a regimen involving lifestyle changes including dietary and exercise regimens. In certain embodiments, the AAV vector and the combination therapy are administered essentially simultaneously. In other embodiments, the AAV vector is administered first. In other embodiments, the combination therapy is delivered first.

In one embodiment, the composition is administered in combination with an effective amount of insulin. Various commercially available insulin products are known in the art, including, without limitation, protamine zinc recombinant human insulin (ProZinc®), porcine insulin zinc suspension (Vetsulin®), insulin glargine (Lantus®), Lispro (Humalog), Aspart (Novolog), Glulisine (Apidra), novolin, and Velosulin.

In some embodiments, combination of the rAAV described herein with insulin decreases insulin dose requirements in the subject, as compared to prior to treatment with the viral vector. Such dose requirements may be reduced by 10% or more, 20% or more, 30% or more, 40% or more, 50% or more, 60% or more, 70% or more, 80% or more, or 90% or more. The treating physician may determine the correct dosage of insulin needed by the subject. For example, the subject may be being treated using insulin or other therapy, which the treating physician may continue upon administration of the AAV vector. Such insulin or other co-therapy may be continued, reduced, or discontinued as needed subsequently.

In one embodiment, composition comprising the expression cassette, vector genome, rAAV, or other composition described herein for gene therapy is delivered as a single dose per patient. In one embodiment, the subject is delivered a therapeutically effective amount of a composition described herein. As used herein, a “therapeutically effective amount” refers to the amount of the expression cassette or vector, or a combination thereof that delivers and expresses in the target cells an amount of GLP1-Fc sufficient to reach therapeutic goal. The therapeutically effective amount may be selected by the treating physician, or guided based on previously determined guidelines. For example, dulaglutide may be provided at an initial dose of 0.75 mg subcutaneously once a week. The dose may be increased in 1.5 mg increments for additional glycemic control. Patients should remain on 1.5 mg once a week dose for at least 4 weeks prior to increasing dose to 3 mg once a week. Patients should remain on 3 mg once a week dose for at least 4 weeks prior to increasing dose to 4.5 mg once a week. The maintenance dose of dulaglutide may be 0.75 to 4.5 mg subcutaneously once a week, with a maximum dose of 4.5 mg weekly. The rAAV may be delivered to the subject and then supplemented with oral or subcutaneous dulaglutide, insulin or other medication as needed to reach the equivalent of the desired dosage of 0.75 to 4.5 mg weekly.

In certain embodiments, the therapeutic goal is to ameliorate or treat one or more of the symptoms of type I diabetes, type II diabetes or metabolic syndrome. A therapeutically effective amount may be determined based on an animal model, rather than a human patient. In another embodiment, the therapeutic goal is remission of the metabolic disease in the subject. As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 20 provides the encoded amino acid sequence of the AAVrh91 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

As used herein, a “stock” of rAAV refers to a population of rAAV. Despite heterogeneity in their capsid proteins due to deamidation, rAAV in a stock are expected to 5 share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected. As used herein the terms “GLP-1 construct”, “GLP-1 expression construct” and synonyms include the GLP-1 sequence as described herein in combination with a leader and fusion domain. The terms “GLP-1 construct”, “GLP-1 expression construct” and synonyms can be used to refer to the nucleic acid sequences encoding the GLP-1 fusion protein or the expression products thereof.

The terms “percent (%) identity”, “sequence identity”, “percent sequence identity”, or “percent identical” in the context of nucleic acid sequences refers to the bases in the two sequences which are the same when aligned for correspondence. The length of sequence identity comparison may be over the full-length of the genome, the full-length of a gene coding sequence, or a fragment of at least about 100 to 150 nucleotides, or as desired. However, identity among smaller fragments, e.g., of at least about nine nucleotides, usually at least about 20 to 24 nucleotides, at least about 28 to 32 nucleotides, at least about 36 or more nucleotides, may also be desired. Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

By the term “highly conserved” is meant at least 80% identity, preferably at least 90% identity, and more preferably, over 97% identity. Identity is readily determined by one of skill in the art by resort to algorithms and computer programs known by those of skill in the art.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example, “95% identity” and “at least 95% identity” may be used interchangeably and include 95%, 96%, 97%, 98%, 99%, and up to 100% identity to the referenced sequence, and all fractions therebetween.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequencers. A suitable amino acid fragment may be at least about 8 amino acids in length, and may be up to about 150 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is not a feline.

As used herein, the term “about” means a variability of 10% (±10%, e.g., ±1, ±2, ±3, ±4, ±5, ±6, ±7, ±8, ±9, ±10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

The term “regulation” or variations thereof as used herein refers to the ability of a composition to inhibit one or more components of a biological pathway.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

A reference to “one embodiment” or “another embodiment” in describing an embodiment does not imply that the referenced embodiment is mutually exclusive with another embodiment (e.g., an embodiment described before the referenced embodiment), unless expressly specified otherwise.

### SPECIFIC EMBODIMENTS

1. A viral vector comprising a nucleic acid comprising a sequence encoding a fusion protein comprising a GLP-1 analog and an IgG4 Fc.
2. The viral vector according to embodiment 1, wherein the vector is an adeno-associated viral vector.
3. The viral vector according to embodiment 1 or embodiment 2, wherein the fusion protein further comprises a thrombin leader sequence.
4. The viral vector according to embodiment 3, wherein the thrombin leader sequence comprises the sequence of SEQ ID NO: 7 or a functional variant thereof having at most 1, 2, or 3 amino acid substitutions.
5. The viral vector according to any one of embodiments 1 to 4, wherein the fusion protein further comprises a spacer.
6. The viral vector according to any one of embodiments 1 to 5, wherein the fusion protein comprises a human thrombin leader, a GLP-1 analog, a spacer, and a human IgG4 Fc.
7. The viral vector according to embodiment 1 to 6, wherein the fusion protein has the sequence of SEQ ID NO: 14, or a sequence at least 99% identical thereto.
8. The viral vector according to any one of embodiments 1 to 7, wherein the sequence encoding the fusion protein is SEQ ID NO: 15
9. The viral vector according to any of embodiments 1 to 8 comprising:
   (a) an AAV capsid, and
   (b) a vector genome packaged in the AAV capsid, said vector genome comprising AAV inverted terminal repeats (ITRs), the coding sequence for the fusion protein, and regulatory sequences which direct expression of the fusion protein.
10. The viral vector according to any one of embodiments 1 to 9, wherein the viral vector is a recombinant adeno-associated virus (rAAV) having the AAV capsid of AAV8 or a functional variant thereof
11. The viral vector according to any one of embodiments 1 to 9, wherein the viral vector is an rAAV having the AAV capsid of AAVrh91 or a functional variant thereof
12. The viral vector according to any one of embodiments 1 to 9, wherein the viral vector is an rAAV having the AAV capsid of AAV3B.AR2.12 or a functional variant thereof
13. The viral vector according to any one of embodiments 1 to 9, wherein the viral vector is an rAAV having the AAV capsid selected from AAV9, AAVrh64R1, AAVhu37, or AAVrh10 or a functional variant thereof
14. The viral vector according to one of embodiments 1 to 13, comprising a vector genome comprising an inducible gene expression system, regulatable promoter, the sequence encoding the fusion protein, and a polyadenylation signal.
15. The viral vector according to any one of embodiments 9 to 14, wherein the AAV inverted terminal repeats (ITRs) are an AAV2 5′ ITR and an AAV2 3′ ITR which flank the fusion protein coding sequence and regulatory sequences.
16. The viral vector according to any one of embodiments 9 to 15, wherein the vector genome comprises a human cytomegalovirus promoter and a rabbit globin poly A.
17. The viral vector according to any one of embodiments 1 to 16, comprising an inducible gene expression system.
18. The viral vector according to embodiment 17, wherein the inducible gene expression system comprises
   (a) an activation domain comprising a transactivation domain and a FKBP12-rapamycin binding (FRB) domain of FKBP12-rapamycin-associated protein (FRAP);
   (b) a DNA binding domain comprising a zinc finger homeodomain (ZFHD) and one, two or three FK506 binding protein domain (FKBP) subunit genes; and
   (c) at least one copy of the binding site for ZFHD followed by a minimal IL2 promoter, and
   (d) a regulatable promoter;
   wherein the presence of an effective amount of a rapamycin or a rapalog induces expression of the transgene in a host cell.
19. The viral vector according to embodiment 18, wherein the FKBP subunit gene sequences share less than about 85% identity with each other.
20. The viral vector according to embodiment 18 or 19, wherein one of the FKBP subunit gene sequences is a native FKBP gene sequence.
21. The viral vector according to any one of embodiments 18 to 20, wherein the transactivation domain comprises a portion of NF-κB p65.
22. The viral vector according to any one of embodiments 18 to 21, wherein the regulatable promoter is a constitutive promoter.
23. The viral vector according to any one of embodiments 18 to 21, wherein the regulatable promoter is a tissue specific promoter.
24. The viral vector according to any one of embodiments 18 to 22, wherein the regulatable promoter is a CMV promoter.
25. The viral vector according to any one of embodiments 18 to 24, further comprising an IRES or 2A.
26. The viral vector according to any one of embodiments 18 to 25, further comprising a 2A linker selected from GT2A_V1 (SEQ ID NO: 21) or GT2A_V2 (SEQ ID NO: 22).
27. The viral vector according to any one of embodiments 18 to 26, comprising at least 8 copies of the binding site for ZFHD.
28. The viral vector according to any one of embodiments 18 to 27, wherein the vector genome comprises a sequence of SEQ ID NO: 16 or a sequence at least 95% to 99.9% identical thereto.
29. A viral vector comprising a nucleic acid molecule comprising: a regulatable promoter; an activation domain comprising a p65 transactivation domain and a FKBP12-rapamycin binding (FRB) domain of FKBP12-rapamycin-associated protein (FRAP); a DNA binding domain comprising a zinc finger homeodomain (ZFHD) and three FK506 binding protein domain (FKBP) subunit genes; 8 copies of the binding site for ZFHD, and a sequence encoding a fusion protein comprising a GLP-1 analog and a human IgG4 Fc.
30. A pharmaceutical composition suitable for use in treating a metabolic disease in a subject comprising an aqueous liquid and the viral vector according to any of embodiments 1 to 20.
31. The pharmaceutical composition according to embodiment 30, wherein the fusion protein comprises a human thrombin leader, a GLP-1 analog, a spacer, and a human IgG4 Fc.
32. The viral vector according to any of embodiments 1 to 29, or the pharmaceutical composition according to any one of embodiments 30 or 31, for use in a method for treating a subject having a metabolic disease.
33. Use of the viral vector according to any of embodiments 1 to 29 or the pharmaceutical composition according to any one of embodiments 29 to 31 in the manufacture of a medicament for treating a subject having a metabolic disease.
34. The viral vector or use according to embodiment 32 or 33, wherein the composition is formulated to be administered a dose of 1×10⁹ GC/kg to 5×10¹³ GC/kg of the rAAV.
35. The viral vector or use according to any one of embodiments 32 or 33, wherein the patient is a human and is administered a dose of 1×10¹⁰ to 1.5×10¹⁵ GC of the rAAV.
36. The viral vector or use according to any one of embodiments 32 to 35, wherein the rAAV is delivered intramuscularly or intravenously.
37. A method of treating a subject having a metabolic disease, comprising delivering to the subject a recombinant adeno-associated virus (rAAV) having an AAV capsid from adeno-associated virus rh91, and a vector genome packaged in the AAV capsid, said vector genome comprising AAV inverted terminal repeats (ITRs), a sequence encoding a fusion protein comprising a GLP-1 analog and a human IgG4 Fc, and regulatory sequences which direct expression of the fusion protein.
38. The method according to embodiment 37, wherein the patient is administered a viral vector according to any of embodiments 1 to 29 or a pharmaceutical composition according to any one of embodiments 30 to 31.
39. The method according to embodiment 37 or 38, wherein the patient is administered a dose of 1×10⁹ GC/kg to 5×10¹³ GC/kg body mass of the rAAV.
40. The method according to any one of embodiments 37 to 39, wherein the rAAV is delivered intramuscularly or intravenously.
41. The viral vector according to any one of embodiments 1 to 29, 32 or 34 to 36, composition according to any one of embodiments 30 to 32, use according to any one embodiments 33 to 36 or method according to any one of embodiments 37 to 40, for treating diabetes in a human.

### EXAMPLES

The following examples are provided to illustrate various embodiments of the present invention. The EXAMPLES are not intended to limit the present invention in any way.

Glucagon like peptide 1 (GLP-1) is a hormone produced from proteolytic cleavage of glucagon preprotein in the gastrointestinal (GI) tract. GLP-1 broadly regulates glucose homeostasis by potentiating insulin release from beta cells, increasing insulin sensitivity of some tissues, slowing gastric emptying (without causing hypoglycemia), and increasing satiety. GLP-1 could not be effectively used as a drug due to its extremely short half-life, but long-acting analogs of GLP-1 have become widely used drugs for the treatment of type 2 diabetes. GLP-1 agonists have an excellent safety profile and require repeated, often life-long parenteral administration, making them good candidates for AAV-mediated gene transfer, which can achieve long term expression following a single administration. GLP-1 and GLP-1 agonists are difficult to express from an AAV vector because the protein cannot be expressed in its native context (the glucagon protein) which requires processing by proteases specific to L cells of the small intestine. Attempts to express GLP-1 using a heterologous signal peptide have failed to achieve high levels of expression. We proposed that signal peptides may not achieve reliable expression because they do not result in appropriate processing of the GLP-1 N-terminus, which is involved in receptor binding. We instead expressed GLP-1 using propeptides, which are cleaved to produce the free GLP-1 protein. We selected propeptides from coagulation factors such as thrombin and factor IX for GLP-1 expression, as these can be cleaved by ubiquitous proteases (e.g., furin) and are endogenous peptides which will not be immunogenic. The thrombin propeptide increased expression of a human GLP-1 analog at least 100-fold relative to a signal peptide alone. Using this technology, we have developed two long acting GLP-1 analogs that can be expressed from an AAV vector; one comprising a IgG4 Fc fusion, and one comprising an albumin fusion, both carrying a human propeptide. We have developed expression cassettes to express these proteins constitutively or in a controlled manner via administration of a small molecule drug that activates transcription of the GLP-1 agonist sequence. The target product profile is designed as a single intramuscular injection. In one embodiment, the single injection comprises an inducible version, as a single pill every 2-4 weeks which is designed to maintain therapeutic GLP-1 agonist levels. As another embodiment, the single injection comprises constitutive version which is designed for continuous lifelong expression at therapeutic levels after one dose. The designed products were testes in preclinical models to examine pharmacology and safety in nonhuman primates. The assays were developed for GLP-1 agonist expression and activity. Safety and pharmacokinetics have been examined to analyze the ability to achieve known therapeutic concentration.

This innovation allows for one-shot, potentially lifelong treatments for type 2 diabetes, especially in patients not achieving glycated hemoglobin (also referred to as glycohemoglobin, hemoglobin A1c, HbA1c, or A1c) goal on metformin alone or other oral agent after 3 months. Standard of care currently includes long-acting subcutaneous GLP-1 agonists, such as liraglutide (administered daily), Dulaglutide (administered weekly), DPP (e.g., Dipeptidyl peptidase-4) IV inhibitors (PO), and Semaglutide PO (administered daily). Prior attempts to achieve AAV-mediated GLP-1 expression either yielded dramatically lower expression, or required use of xenogenic leader sequences that would be immunogenic and unsuitable for clinical applications.

### Example 1—Construction of GLP-1 Vectors

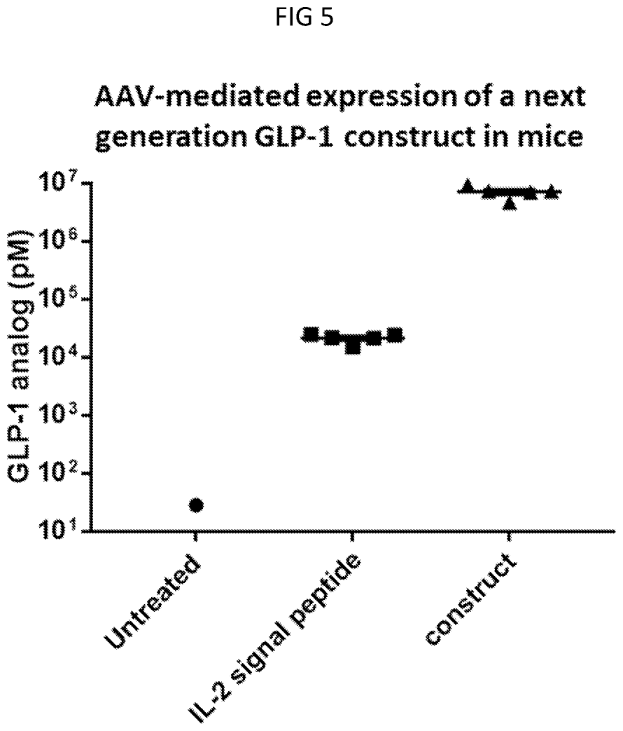

GLP-1 agonists are challenging to express via adeno-associated virus (AAV). GLP-1 is normally expressed from the glucagon precursor protein, which requires tissue specific proteases and produces unwanted proteins. Expression systems using traditional heterologous signal peptides yield low expression. Expression systems using heterologous propeptides with universal protease cleavage sites yield foreign protein sequences that could be targets for T cells. We developed a system that increases GLP-1 expression about 300-fold from liver or muscle cells without introducing foreign protein sequences. FIG. 5 shows an AAV-mediated expression of an engineered GLP-1 construct in mice. Mice received an intramuscular injection of an AAV vector expressing a GLP-1 agonist with a standard IL-2 signal peptide or an endogenous precursor which we have developed. Serum GLP-1 concentration was measured by ELISA 3 weeks after injection.

More specifically, vectors were constructed in which a leader sequence was placed upstream of one of several GLP-1 receptor agonist amino acid sequences followed by a fusion domain. See, e.g., FIG. 4. The resulting protein sequence was back-translated, followed by addition of a kozak consensus sequence, stop codon, and cloning sites. The sequences were produced, and cloned into an expression vector containing a CMV promoter under the control of an inducible expression system. The expression construct was flanked by AAV2 ITRs. The resulting plasmid is called pAAV.TF.GT2A.dulaglutide(trb).3w.rBG. The human thrombin-dulaglutide amino acid sequence is shown in SEQ ID NO: 14; the coding sequence is shown in SEQ ID NO: 15; the vector genome is shown in SEQ ID NO: 16.

Currently available inducible constructs include a 2-vector and a 1-vecotr inducible systems. See, e.g., FIG. 6A and FIG. 6B. FIG. 6A shows a schematic of an example expression cassette comprising inducible construct for use in a two-vector system. FIG. 6B shows a schematic of an expression cassette comprising an inducible construct for use in a 1-vector system, comprising an IRES linker.

Furthermore, we introduced a GT2A peptide in the expression vector comprising GLP1-Fc transgene. Human GLP1-Fc (hDulaglutide) with secretory signal is 954 bp. For expression of hDulaglutide construct, as described above, in an expression vector as shown in FIG. 6B, we replaced an IRES linker with a GT2A cleavage sequence, which allows it to fit in the packaging limit (FIG. 7A; single inducible cassette for GLP-1 Fc). The GT2A peptide is selected from GT2A_V1 peptide comprising amino acid sequence of SEQ ID NO: 21, or GT2A_V2 peptide comprising amino acid sequence of SEQ ID NO: 22. FIG. 7A shows a schematic of an expression cassette comprising an inducible construct for use in a 1-vector system, comprising an F2A cleavage sequence linker and human GLP1-Fc (hDulaglutide) with secretory signal.

### Example 2—In Vitro Expression

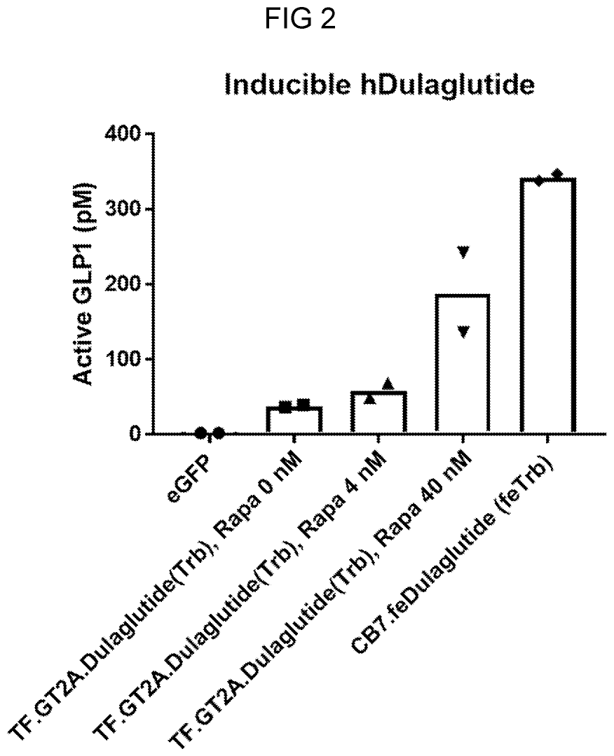

GLP1-Fc fusions were measured in culture supernatants of HEK293 cells transfected with plasmids for inducible human Dulaglutide with human Thrombin signal sequence (TF.GT2A.Dulaglutide(Trb)) and CB7.feline Dulaglutide (feTrb). By feline Dulaglutide is meant a construct where the IgG Fc portion of dulaglutide is replaced with a feline IgG sequence, optionally in combination with a feline thrombin leader (feTrb). Supernatants were collected at 48 hr after treatment with Rapamycin (Rapa) at 0, 4, and 40 nM or at 48 hr after transfection for CB7.feDulaglutide(feTrb). GLP1-Fc was quantified by active form GLP1 ELISA along with kit's STD. The expression of the three constructs is shown in FIG. 2. Increasing dosages of rapamycin led to increasing expression of GLP-1.

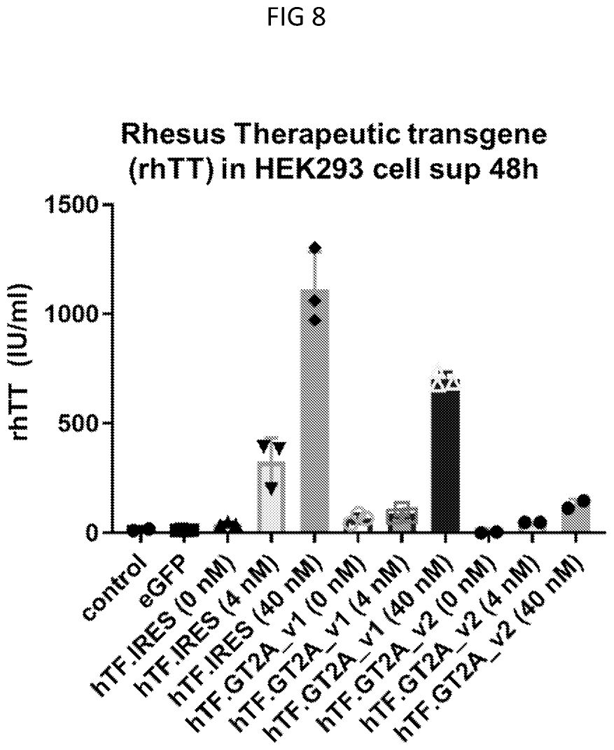

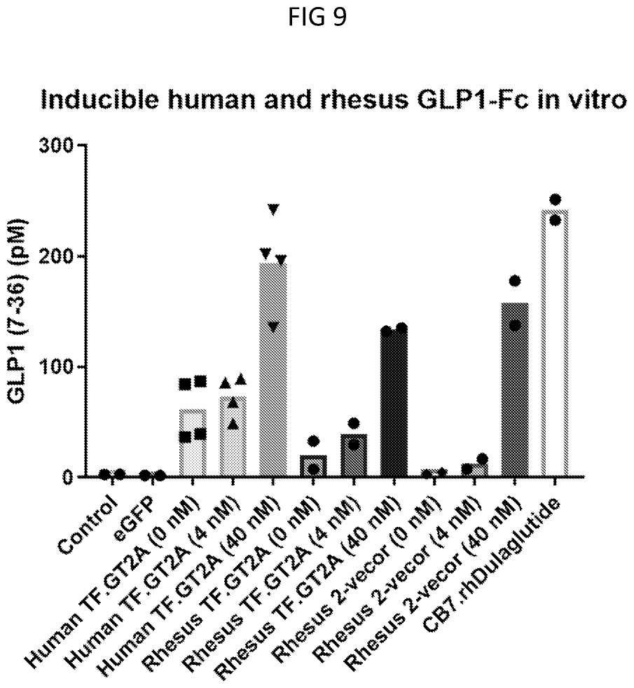

Furthermore, we evaluated expression of a rhesus macaque exemplary therapeutic transgene (rhTT) in the designed constructs comprising GT2A_V1 or GT2A_V2 peptides (FIGS. 6B, 7A and 7B). FIG. 8 shows expression of rhesus monkey therapeutic transgene (rhTT) in HEK293 cell supernatant as measured following transfection with various constructs comprising GT2A peptide and treatment with Rapamycin at 0 nM, 4 nM, and 40 nM, and plotted as IU/mL of rhTT. Next, we examined expression of human and rhesus macaque GLP-1 Fc expression in vitro using the designed single inducible cassette comprising GT2A_V1 and GT2A_V2 peptides. FIG. 9 shows inducible human (h) and rhesus macaque (rh) GLP-1 expression in vitro. GLP1-Fc fusions were measured in culture supernatants of HEK293 cells transfected with plasmids for inducible hDulaglutide comprising Thrombin signal sequence, rhDulaglutide comprising 2-vector system, and CB7.rhDulaglutide. Cell were plated on Day 0, transfected in Day 1, treated with Rapamycin at 0 nM, 4 nM, and 40 nM on Day 2, and supernatants from cells were collected on Day 4 or at 48 hr after transfection for CB7.rhDulaglutide(rhTrb). GLP1-Fc was quantified by active form GLP1 ELISA along with kit's STD.

### Example 3—Pilot Expression in Rag1KO Mice

The following constructs were packaged in an AAVrh91 vector by triple transfection and iodixanol gradient purification, as previously described.

AAVrh91.TF.hDulaglutide(Trb).3w.rBG, with human thrombin signal

AAVrh91.TF.rhDulaglutide(rhTrb).3w.rBG, with rhesus thrombin signal

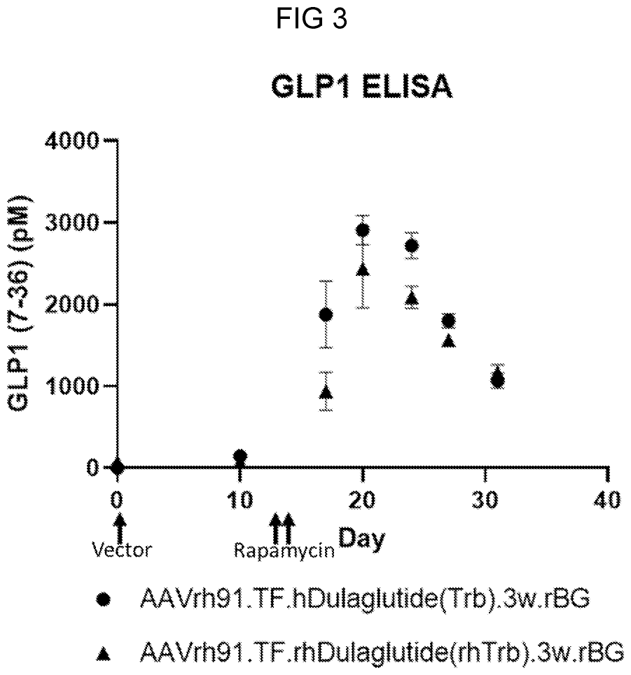

Rag1KO female mice (n=5/vector) were treated with an injection of the vector (1×10¹¹ GC/mouse) via IM route of administration. Serum was serially collected by separating whole blood in serum separator tubes containing 5 microliters DPP-IV inhibitor (Millipore) and assayed for active GLP-1 expression and activity as above. Vector was injected at day 0 and rapamycin administered around day 14 and 15. Serum active GLP-1 concentrations are shown in FIG. 3. Serum levels reached maximum value approximately 1 week post rapamycin administration.

### Example 4—Long-Term Expression Study in NHPs

In this study, we examined expression of rhesus macaque GLP-1 (rhDulaglutide) in nonhuman primates (NHPs; i.e., rhesus macaques). Tables 1A and 1B shows an outline of a study including AAV administration and Rapamycin administration (i.e., induction). Briefly, NHPs1-3 were administered AAVrh91 designated vectors via intramuscular injection (IM)—NHP1: AAVrh91.CB7.rhDulaglutide.rBG at a dose of 1×10¹² (1e12) GC/kg; NHP2: AAVrh91.CMV.TFNc.3 AAVrh91.Z12I.rhDulaglutide.rBG and AAVrh91.Z12I.rhDulaglutide.rBG at a dose 5×10¹² (5e12) GC/kg each; and NHP3: 1×10¹³ (1e13) GC/kg. For NHP2, rapamycin was administered at day 21 at a dose of 0.5 mg/kg, day 56 at a dose of 0.5 mg/kg, and day 126 at a dose of 2.0 mg/kg. For NHP3, rapamycin was administered, at day 21 at a dose of 0.5 mg/kg, day 78 at a dose of 0.5 mg/kg, and at day 148 at a dose of 2.0 mg/kg.

TABLE 1A

| Animal ID | Vector | Dose | Route |
|---|---|---|---|
| NHP1 (18-128) | AAVrh91.CB7.rhDulaglutide.rBG | 1e12 GC/kg | IM |
| NHP2 (18-072) | AAVrh91.CMV.TFNc.3 | 5e12 GC/kg each | IM |
| | AAVrh91.Z12I.rhDulaglutide.rBG | | |
| NHP3 (18-013) | AAVrh91.TF.GT2A.rhDulaglutide.rBG | 1e13 GC/kg | IM |

TABLE 1B

| Induction | NHP2 (18-072) | NHP3(18-013) |
|---|---|---|
| 1st: Rapamycin IV, 0.5 mg/kg | Day 21 | Day 21 |
| 2nd: Rapamycin IV, 0.5 mg/kg | Day 56 | Day 78 |
| 3rd: Rapamycin PO, 2.0 mg/kg | Day 126 | Day 148 |

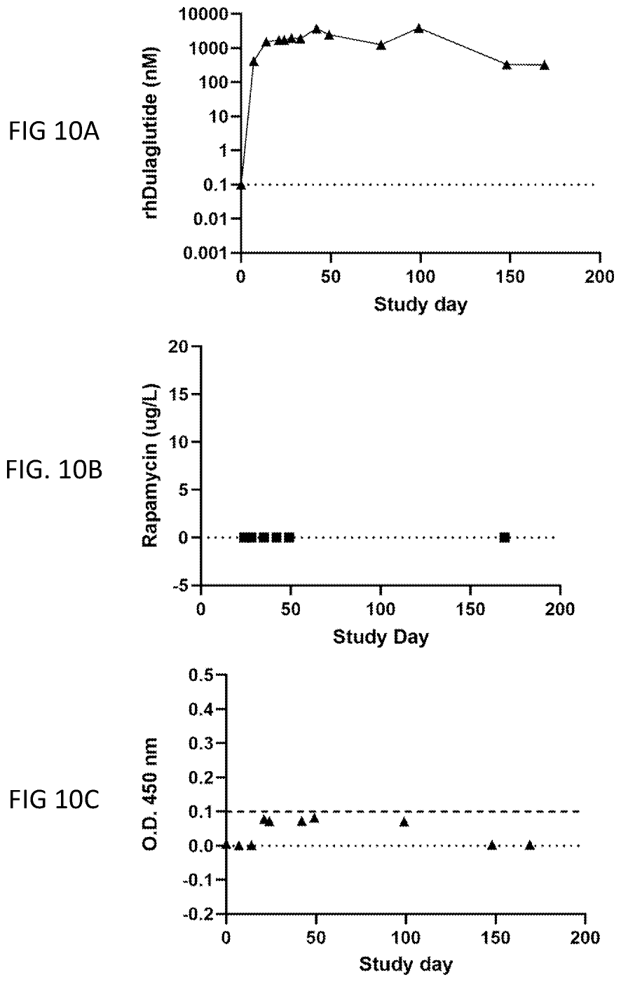

FIGS. 10A to 10C show rhGLP1-Fc expression and analysis of an anti-rhGLP1-Fc ADA (anti-drug antibody) detection assay for NHP1 (18-128). FIG. 10A shows rhGLP1-Fc expression levels in serum plotted as nM, as measured on days 0 to 200. FIG. 10B shows rapamycin levels in serum plotted as μg/L, as measured on days 0 to 200. FIG. 10C shows results of an ADA detection assay plotted as O.D. 450 nm, as measured on days 0 to 200.

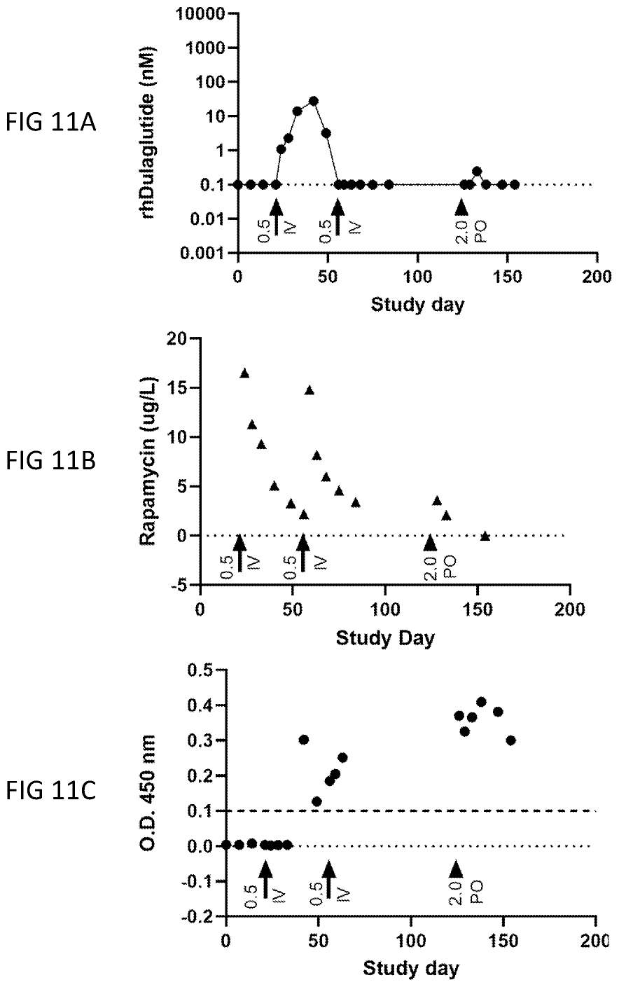

FIGS. 11A to 11C show rhGLP1-Fc expression and analysis of an anti-rhGLP1-Fc ADA assay for NHP1 (18-072). FIG. 11A shows rhGLP1-Fc expression levels in serum plotted as nM, as measured on days 0 to 200. FIG. 11B shows rapamycin levels in serum plotted as μg/L, as measured on days 0 to 200. FIG. 11C shows results of an ADA detection assay plotted as O.D. 450 nm, as measured on days 0 to 200.

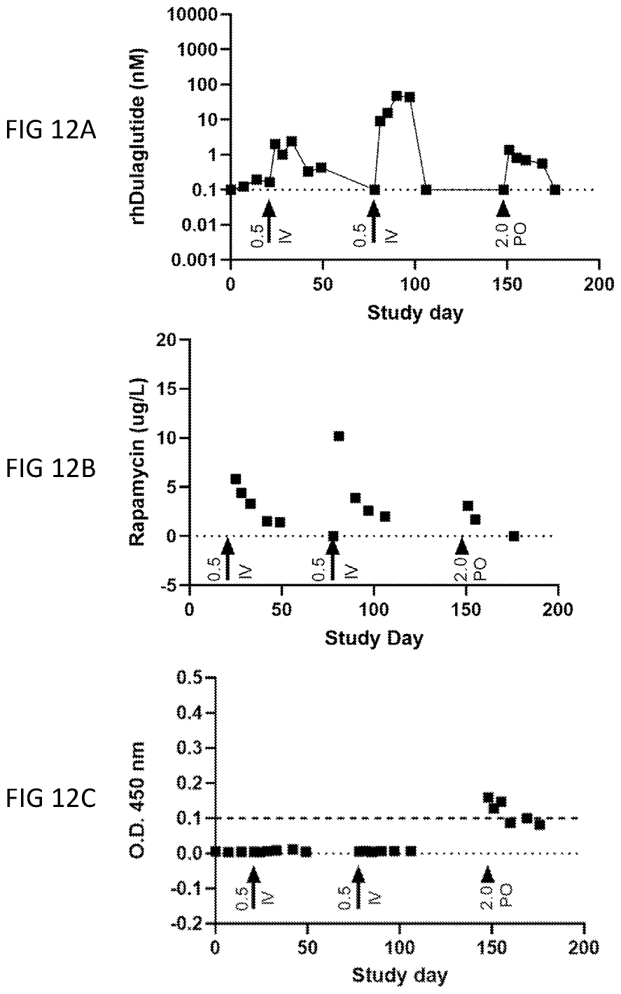

FIGS. 12A to 12C show rhGLP1-Fc expression and analysis of an anti-rhGLP1-Fc ADA assay for NHP1 (18-013). FIG. 12A shows rhGLP1-Fc expression levels in serum plotted as nM, as measured on days 0 to 200. FIG. 12B shows rapamycin levels in serum plotted as μg/L, as measured on days 0 to 200. FIG. 12C shows results of an ADA detection assay plotted as O.D. 450 nm, as measured on days 0 to 200.

In summary, we have developed a 1-vector inducible system for expression of human GLP1-Fc fusion. Additionally, we confirmed induction of human GLP1-Fc upon rapamycin in Rag1KO mice. In NHPs, we observed that 1-vector and 2-vector inducible vectors expressing monkey GLP1-Fc respond to rapamycin and lead to a transient increase of serum GLP1-Fc with greater than 1 nM with more than 20 days duration. We observed that a low dose constitutively expressing vector provided high and sustained expression of serum GLP1-Fc in an NHP.

(Sequence Listing Free Test)
The following information is provided for sequences
containing free text under numeric identifier <223>.

| SEQ ID NO: | |
|---|---|
| (containing | |
| free text) | Free text under <223> |
| 3 | <223> Constructed sequence |
| 4 | <223> Constructed sequence |
| 5 | <223> Constructed sequence |
| 6 | <223> Constructed sequence |
| 13 | <223> Constructed sequence |
| 14 | <223> Constructed sequence |
| 15 | <223> Constructed sequence |
| 16 | <223> Constructed sequence |
| 18 | <220> |
| | <221> misc feature |
| | <222> (1) . . . (2211) |
| | <223> AAVrh91 |
| 21 | <220> |
| | <221> SITE |
| | <222> (20) . . . (21) |
| | <223> Cleavage |
| 22 | <220> |
| | <221> SITE |
| | <222> (20) . . . (21) |
| | <223> Cleavage |
| 23 | <223> nucleic acid sequecne of FKBP12- |
| | rapamycin binding (FRB) domain of human |
| | FKBP12-rapamycin-associated protein (FRAP) |
| 24 | <223> amino acid sequence of FKBP12- |
| | rapamycin binding (FRB) domain of human |
| | FKBP12-rapamycin-associated protein (FRAP) |
| 25 | <223> nucleic acid sequence p65 subunit of |
| | NF-kappa B from human |
| 26 | <223> amino acid sequence p65 subunit of NF- |
| | kappa B from human |
| 27 | <223> CMV promoter |
| 28 | <223> nucleic acid sequence for zinc finger |
| | homeodomain (ZFHD1) |
| 29 | <223> amino acid sequence for zinc finger |
| | homeodomain (ZFHD1) |
| 30 | <223> nucleic acid sequence for FK506 |
| | binding protein domain (FKBP) subunit genes |
| 31 | <223> amino acid sequence for FK506 binding |
| | protein domain (FKBP) subunit genes |
| 32 | <223> binding site for ZFHD |
| 33 | <223> constructed sequence |
| 34 | <223> constructed sequence |
| 35 | <223> constructed sequence |
| 38 | <223> constructed sequence |
| 39 | <223> constructed sequence |
| 40 | <223> constructed sequence |
| 41 | <223> constructed sequence |

All documents cited in this specification, are incorporated herein by reference. U.S. provisional Patent Application No. 63/069,500, filed Aug. 24, 2020 is incorporated herein by reference in its entireties, together with its sequence listing. The sequence listing filed herewith labeled “20-9429PCT_Seq_List_ST25” and the sequences and the text therein are incorporated by reference. While the invention has been described with reference to particular embodiments, it will be appreciated that modifications can be made without departing from the spirit of the invention. Such modifications are intended to fall within the scope of the appended claims.

1. A viral vector comprising a nucleic acid comprising a sequence encoding a fusion protein comprising a GLP-1 analog and an IgG4 Fc, wherein the fusion protein has the sequence of SEQ ID NO: 14, or a sequence at least 99% identical thereto.

2. The viral vector of claim 1, wherein the sequence encoding the fusion protein is SEQ ID NO: 15, or a sequence sharing at least 75% identical thereto.

3. The viral vector of claim 1 comprising:
(a) an AAV capsid, and
(b) a vector genome packaged in the AAV capsid, said vector genome comprising AAV inverted terminal repeats (ITRs), the coding sequence for the fusion protein, and regulatory sequences which direct expression of the fusion protein.

4. The viral vector of claim 1, wherein the viral vector is an rAAV having the AAV capsid of AAVrh91.

5. The viral vector according to of claim 1, comprising a vector genome comprising an inducible gene expression system, regulatable promoter, the sequence encoding the fusion protein, and a polyadenylation signal.

6. The viral vector according to claim 3, wherein the AAV inverted terminal repeats (ITRs) are an AAV2 5′ ITR and an AAV2 3′ ITR which flank the fusion protein coding sequence and regulatory sequences.

7. The viral vector of claim 5, wherein the vector genome comprises a CB7 promoter and a rabbit globin poly A.

8. (canceled)

9. The viral vector of claim 5, wherein the inducible gene expression system comprises
(a) an activation domain comprising a transactivation domain and a FKBP12-rapamycin binding (FRB) domain of FKBP12-rapamycin-associated protein (FRAP);
(b) a DNA binding domain comprising a zinc finger homeodomain (ZFHD) and one, two or three FK506 binding protein domain (FKBP) subunit genes; and
(c) at least one copy of the binding site for ZFHD followed by a minimal IL2 promoter, and
(d) a regulatable promoter;
wherein the presence of an effective amount of a rapamycin or a rapalog induces expression of the transgene in a host cell.

10. The viral vector of claim 9, wherein the FKBP subunit gene sequences share less than about 85% identity with each other.

11. The viral vector of claim 9, wherein one of the FKBP subunit gene sequences is a native FKBP gene sequence.

12. The viral vector of claim 9, wherein the transactivation domain comprises a portion of NF-κB p65.

13. The viral vector of claim 9, wherein the regulatable promoter is a constitutive promoter.

14. The viral vector of claim 9, wherein the regulatable promoter is a CMV promoter.

15. The viral vector of claim 9, further comprising an IRES or 2A.

16. The viral vector of claim 9, further comprising a 2A linker selected from GT2A_V1 (SEQ ID NO: 21) or GT2A_V2 (SEQ ID NO: 22).

17. The viral vector according of claim 9, comprising at least 8 copies of the binding site for ZFHD.

18. The viral vector of claim 9, wherein the vector genome comprises a sequence of SEQ ID NO: 16 or a sequence at least 70% identical thereto.

19. A viral vector comprising a nucleic acid molecule comprising: a regulatable promoter; an activation domain comprising a p65 transactivation domain and a FKBP12-rapamycin binding (FRB) domain of FKBP12-rapamycin-associated protein (FRAP); a DNA binding domain comprising a zinc finger homeodomain (ZFHD) and three FK506 binding protein domain (FKBP) subunit genes; 8 copies of the binding site for ZFHD, and a sequence encoding a fusion protein comprising a GLP-1 analog and a human IgG4 Fc.

20. A pharmaceutical composition suitable for use in treating a metabolic disease in a subject comprising an aqueous liquid and the viral vector of claim 1.

21-25. (canceled)

26. A method of treating a subject having a metabolic disease, comprising delivering to the subject a recombinant adeno-associated virus (rAAV) having an AAV capsid from adeno-associated virus rh91, and a vector genome packaged in the AAV capsid, said vector genome comprising AAV inverted terminal repeats (ITRs), a sequence encoding a fusion protein comprising a GLP-1 analog and a human IgG4 Fc, and regulatory sequences which direct expression of the fusion protein.

27-30. (canceled)


SEQUENCE LISTING (41 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 31 aa, Homo sapiens
HAEGTFTSDVSSYLEGQAAKEFIAWLVKGRG

SEQ ID NO: 3 - protein, 31 aa, Artificial Sequence
  Constructed sequence
HGEGTFTSDVSSYLEEQAAKEFIAWLVKGGG

SEQ ID NO: 4 - protein, 31 aa, Artificial Sequence
  Constructed sequence
HGEGTFTSDVSSYLEGQAAKEFIAWLVKGRG

SEQ ID NO: 5 - protein, 39 aa, Artificial Sequence
  Constructed sequence
HGEGTFTSDLSKQMEEEAVRLFIEWLKNGGPSSGAPPPS

SEQ ID NO: 6 - protein, 44 aa, Artificial Sequence
  Constructed sequence
HGEGTFTSDLSKQMEEEAVRLFIEWLKNGGPSSGAPPSKKKKKK

SEQ ID NO: 7 - protein, 43 aa, Homo sapiens
MAHVRGLQLPGCLALAALCSLVHSQHVFLAPQQARSLLQRVRR

SEQ ID NO: 8 - protein, 24 aa, Homo sapiens
MAHVRGLQLPGCLALAALCSLVHS

SEQ ID NO: 9 - protein, 19 aa, Homo sapiens
QHVFLAPQQARSLLQRVRR

SEQ ID NO: 10 - protein, 20 aa, Homo sapiens
MYRMQLLSCIALSLALVTNS

SEQ ID NO: 11 - protein, 229 aa, Homo sapiens
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 585 aa, Homo sapiens [sequence omitted]

SEQ ID NO: 13 - protein, 15 aa, Artificial Sequence
  Constructed sequence
GGGGSGGGGSGGGGS

SEQ ID NO: 14 - protein, 318 aa, Artificial Sequence
  Constructed sequence
MAHVRGLQLPGCLALAALCSLVHSQHVFLAPQQARSLLQRVRRHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - DNA, 954 bp, Artificial Sequence
  Constructed sequence
atggctcacgttcgaggactgcagctgcctggatgtctggctcttgccgctctgtgtagcctggtgcacagccagcacgtgtttctggctcctcagcaagccagatcactgctgcagagagttagaaggcacggcgagggcacctttacctccgacgtgtctagctacctggaagaacaggccgccaaagagtttatcgcctggctggtcaaaggtggcggcggaggcggaggaagcggtggcggaggttcaggtggtggtggatctgccgagtctaagtacggccctccttgtcctccctgtcctgctcccgaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagctccatcgagaaaaccatcagcaaggccaagggccagccaagagaaccccaggtgtacacactgcctccaagccaagaggaaatgaccaagaaccaggtgtccctgacctgcctcgtgaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggc

SEQ ID NO: 16 - DNA, 4931 bp, Artificial Sequence
  constructed sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagtacgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggctctggcgaaggcagaggcagcctgcttacctgtggcgacgtggaagagaaccccggacctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagagagttagaaggcacggcgagggcacctttacctccgacgtgtctagctacctggaagaacaggccgccaaagagtttatcgcctggctggtcaaaggtggcggcggaggcggaggaagcggtggcggaggttcaggtggtggtggatctgccgagtctaagtacggccctccttgtcctccctgtcctgctcccgaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagctccatcgagaaaaccatcagcaaggccaagggccagccaagagaaccccaggtgtacacactgcctccaagccaagaggaaatgaccaagaaccaggtgtccctgacctgcctcgtgaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggctaaggtaccgatctttttccctctgccaaaaattatggggacatcatgaagccccttgagcatctgacttctggctaataaaggaaatttattttcattgcaatagtgtgttggaattttttgtgtctctcactcggaaggcgcgccgatcctcgagactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 17 - protein, 224 aa, Macaca mulatta
PPCPPCPAPELLGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVMHEALHNHYTQKSLSVSPGK

SEQ ID NO: 18 - DNA, 2211 bp, adeno-associated virus rh91
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 19 - DNA, 2211 bp, adeno-associated virus rh91
  misc_feature (1)..(2211): AAVrh.91
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 20 - protein, 736 aa, adeno-associated virus rh91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 21 - protein, 29 aa, Constructed sequence
  SITE (20)..(21): Cleavage
GSGEGRGSLLTCGDVEENPGPAAKRVKLD

SEQ ID NO: 22 - protein, 33 aa, Constructed sequence
  SITE (20)..(21): Cleavage
GSGEGRGSLLTCGDVEENPGPMYDPAAKRVKLD

SEQ ID NO: 23 - DNA, 279 bp, Artificial Sequence
  nucleic acid sequecne of FKBP12-rapamycin binding (FRB) domain of human FKBP12-rapamycin-associated protein (FRAP)
atggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaa

SEQ ID NO: 24 - protein, 93 aa, Artificial Sequence
  amino acid sequence of FKBP12-rapamycin binding (FRB) domain of human FKBP12-rapamycin-associated protein (FRAP)
MDYPAAKRVKLDSRILWHEMWHEGLEEASRLYFGERNVKGMFEVLEPLHAMMERGPQTLKETSFNQAYGRDLMEAQEWCRKYMKSGNVKDLLQ

SEQ ID NO: 25 - DNA, 573 bp, Artificial Sequence
  nucleic acid sequence p65 subunit of NF-kappa B from human
gatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 26 - protein, 191 aa, Artificial Sequence
  amino acid sequence p65 subunit of NF-kappa B from human
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 27 - DNA, 672 bp, Artificial Sequence
  CMV promoter
acgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccacc

SEQ ID NO: 28 - DNA, 402 bp, Artificial Sequence
  nucleic acid sequence for zinc finger homeodomain (ZFHD1)
cctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactaga

SEQ ID NO: 29 - protein, 134 aa, Artificial Sequence
  amino acid sequence for zinc finger homeodomain (ZFHD1)
PAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTR

SEQ ID NO: 30 - DNA, 978 bp, Artificial Sequence
  nucleic acid sequence for FK506 binding protein domain (FKBP) subunit genes
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtga

SEQ ID NO: 31 - protein, 107 aa, Artificial Sequence
  amino acid sequence for FK506 binding protein domain (FKBP) subunit genes
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 32 - DNA, 18 bp, Artificial Sequence
  binding site for ZFHD
aatgatgggcgctcgagt

SEQ ID NO: 33 - DNA, 665 bp, Artificial Sequence
  constructed sequence
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 34 - DNA, 2903 bp, Artificial Sequence
  constructed sequence
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagagagttagaaggcacggcgagggcacctttacctccgacgtgtctagctacctggaagaacaggccgccaaagagtttatcgcctggctggtcaaaggtggcggcggaggcggaggaagcggtggcggaggttcaggtggtggtggatctgccgagtctaagtacggccctccttgtcctccctgtcctgctcccgaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagctccatcgagaaaaccatcagcaaggccaagggccagccaagagaaccccaggtgtacacactgcctccaagccaagaggaaatgaccaagaaccaggtgtccctgacctgcctcgtgaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggctaaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 35 - DNA, 2897 bp, Artificial Sequence
  constructed sequence
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccctgcagcgggaattgccaccatggctcacgttcgaggactgcagctgcctggatgtctggctcttgccgctctgtgtagcctggtgcacagccagcatgtgtttctggctcctcaacaagccctgagcctgctgcaaagagttagaaggcacggcgagggcaccttcacctccgacgtgtccagctacctggaagaacaggccgccaaagagtttatcgcctggctggtcaaaggcggtggtggtggcggaggatctggcggaggtggaagcggcggaggcggatctgctgagtttacacctccttgtcctccctgtcctgctcccgagctgctcggaggcccttccgtgtttctgttccctccaaagcctaaggacaccctgatgatcagcagaacccctgaagtgacctgcgtggtcgtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggaatgggagagcaacggacagcccgagaacacctacaagaccacacctccagtgctggacagcgacggcagctatctgctgtactccaagctgacagtgaacaagagccggtggcagcccggcaacatcttcacctgttctgtgatgcacgaggccctgcacaaccactacacccagaagtctctgagcgtcagccctggctaaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 36 - DNA, 951 bp, Macaca mulatta
  CDS (1)..(951)
atggctcacgttcgaggactgcagctgcctggatgtctggctcttgccgctctgtgtagcctggtgcacagccagcatgtgtttctggctcctcaacaagccctgagcctgctgcaaagagttagaaggcacggcgagggcaccttcacctccgacgtgtccagctacctggaagaacaggccgccaaagagtttatcgcctggctggtcaaaggcggtggtggtggcggaggatctggcggaggtggaagcggcggaggcggatctgctgagtttacacctccttgtcctccctgtcctgctcccgagctgctcggaggcccttccgtgtttctgttccctccaaagcctaaggacaccctgatgatcagcagaacccctgaagtgacctgcgtggtcgtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggaatgggagagcaacggacagcccgagaacacctacaagaccacacctccagtgctggacagcgacggcagctatctgctgtactccaagctgacagtgaacaagagccggtggcagcccggcaacatcttcacctgttctgtgatgcacgaggccctgcacaaccactacacccagaagtctctgagcgtcagccctggctaa

SEQ ID NO: 37 - protein, 316 aa, Macaca mulatta
MAHVRGLQLPGCLALAALCSLVHSQHVFLAPQQALSLLQRVRRHGEGTFTSDVSSYLEEQAAKEFIAWLVKGGGGGGGSGGGGSGGGGSAEFTPPCPPCPAPELLGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVMHEALHNHYTQKSLSVSPG

SEQ ID NO: 38 - DNA, 4545 bp, Artificial Sequence
  constructed sequence
acgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggctctggcgaaggcagaggcagcctgcttacctgtggcgacgtggaagagaaccccggacctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgaattcgccctgcagcgggaattgccaccatggctcacgttcgaggactgcagctgcctggatgtctggctcttgccgctctgtgtagcctggtgcacagccagcacgtgtttctggctcctcagcaagccagatcactgctgcagagagttagaaggcacggcgagggcacctttacctccgacgtgtctagctacctggaagaacaggccgccaaagagtttatcgcctggctggtcaaaggtggcggcggaggcggaggaagcggtggcggaggttcaggtggtggtggatctgccgagtctaagtacggccctccttgtcctccctgtcctgctcccgaagctgctggcggcccatccgtgtttctgttccctccaaagcctaaggacaccctgatgatcagcagaacccctgaagtgacctgcgtggtggtcgacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagctccatcgagaaaaccatcagcaaggccaagggccagccaagagaaccccaggtgtacacactgcctccaagccaagaggaaatgaccaagaaccaggtgtccctgacctgcctcgtgaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctgggctaaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 39 - DNA, 4539 bp, Artificial Sequence
  constructed sequence
acgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatcttcggccagcgaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccggctctggcgaaggcagaggcagcctgcttacctgtggcgacgtggaagagaaccccggacctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggcgttcaggtggaaaccatcagtccaggggatggccgaacttttccaaagagagggcagacttgcgtcgtgcattatactggtatgctggaggatgggaaaaagttcgactcttccagagatcggaacaaaccattcaaattcatgctcgggaaacaggaagttatccgcggatgggaggagggcgtggcccagatgtccgtgggccagcgcgccaagctaaccatctccccagactacgcctacggagccaccggacaccccggtatcatacccccacacgccacccttgtgtttgacgtggaactgcttaagctagagactagaggcgtgcaggtcgagaccatcagccccggcgacggccgcacctttcccaagagaggccagacttgcgtggtccactacaccggcatgctggaggacggcaagaagttcgacagcagccgcgaccgcaacaagcccttcaagttcatgctgggcaaacaggaagtgatccgcggctgggaggaaggcgtggctcagatgagcgtggggcagcgggccaagctgaccatcagccccgactatgcctacggcgccaccggccaccccggcatcatccccccccacgccaccctggtgttcgacgtggagctgctgaagctggagtgataaggatccccactccagtgcccaccagccttgtcctaataaaattaagttgcatcattttgtctgactaggtgtccttctataatattatggggtggaggggggtggtttggagcaaggcgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgaattcgccctgcagcgggaattgccaccatggctcacgttcgaggactgcagctgcctggatgtctggctcttgccgctctgtgtagcctggtgcacagccagcatgtgtttctggctcctcaacaagccctgagcctgctgcaaagagttagaaggcacggcgagggcaccttcacctccgacgtgtccagctacctggaagaacaggccgccaaagagtttatcgcctggctggtcaaaggcggtggtggtggcggaggatctggcggaggtggaagcggcggaggcggatctgctgagtttacacctccttgtcctccctgtcctgctcccgagctgctcggaggcccttccgtgtttctgttccctccaaagcctaaggacaccctgatgatcagcagaacccctgaagtgacctgcgtggtcgtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggaatgggagagcaacggacagcccgagaacacctacaagaccacacctccagtgctggacagcgacggcagctatctgctgtactccaagctgacagtgaacaagagccggtggcagcccggcaacatcttcacctgttctgtgatgcacgaggccctgcacaaccactacacccagaagtctctgagcgtcagccctggctaaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 40 - DNA, 1492 bp, Artificial Sequence
  constructed sequence
acgcgtgctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagctaatgatgggcgctcgagtaatgatgggcggtcgactaatgatgggcgctcgagtaatgatgggcgtctagaacgcgaattaattcaacattttgacacccccataatatttttccagaattaacagtataaattgcatctcttgttcaagagttccctatcactctctttaatcactactcacagtaacctcaactcctgccacaagcttgccctgcagcgggaattcgccctgcagcgggaattgccaccatggctcacgttcgaggactgcagctgcctggatgtctggctcttgccgctctgtgtagcctggtgcacagccagcatgtgtttctggctcctcaacaagccctgagcctgctgcaaagagttagaaggcacggcgagggcaccttcacctccgacgtgtccagctacctggaagaacaggccgccaaagagtttatcgcctggctggtcaaaggcggtggtggtggcggaggatctggcggaggtggaagcggcggaggcggatctgctgagtttacacctccttgtcctccctgtcctgctcccgagctgctcggaggcccttccgtgtttctgttccctccaaagcctaaggacaccctgatgatcagcagaacccctgaagtgacctgcgtggtcgtggacgtgtcccaagaggatcctgaggtgcagttcaattggtacgtggacggcgtggaagtgcacaacgcccagacaaagcccagagagcggcagttcaacagcacctacagagtggtgtccgtgctgaccgtgacacaccaggattggctgaacggcaaagagtacacctgtaaagtctccaacaagggcctgcctgctcctatcgagaaaaccatcagcaaggccaagggccagcctagagaaccccaggtgtacatcctgcctccacctcaagaggaactgaccaagaaccaggtgtccctgacctgtctggtcaccggcttctacccttccgatatcgccgtggaatgggagagcaacggacagcccgagaacacctacaagaccacacctccagtgctggacagcgacggcagctatctgctgtactccaagctgacagtgaacaagagccggtggcagcccggcaacatcttcacctgttctgtgatgcacgaggccctgcacaaccactacacccagaagtctctgagcgtcagccctggctaaggtaccgatctttttccctctgccaaaaattatggggacatcatgaagccccttgagcatctgacttctggctaataaaggaaatttattttcattgcaatagtgtgttggaattttttgtgtctctcactcg

SEQ ID NO: 41 - DNA, 3856 bp, Artificial Sequence
  constructed sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcagatctgcttcagctggaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgcagctgctgcagcgggaattccagaagccaccatggactatcctgctgccaagagggtcaagttggactctagaatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcctccaagcctgggacctctattatcatgtgttccgacgaatctcaaagactagagatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctagtaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggactatcctgctgccaagagggtcaagttggactctagagaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcggcggccgcaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagaggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatagtaaggatcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattcgggaaccaagctgg